(19) United States
(12) Patent Application Publication
(10) Pub. No.: US 2022/0189598 A1
(43) Pub. Date: Jun. 16, 2022

(54) SYSTEM AND METHOD OF TUBERCULOSIS THERAPY

(71) Applicant: BAYLOR RESEARCH INSTITUTE, Dallas, TX (US)

(72) Inventors: Tawanda GUMBO, Dallas, TX (US); Magombedze GESHAM, Dallas, TX (US)

(21) Appl. No.: 17/310,556

(22) PCT Filed: Feb. 12, 2020
(86) PCT No.: PCT/US2020/017850

§ 371 Date: Aug. 10, 2021

Related U.S. Application Data
(60) Provisional application No. 62/804,590, filed on Feb. 12, 2019.

(51) Int. Cl. G16H 20/10; C12Q 1/06; G16H 10/60

(52) U.S. Cl. CPC G16H 20/10; G16H 10/60; C12Q 1/06

(57) ABSTRACT

The present disclosure relates to methods, devices, and systems for treating a patient with tuberculosis. In an aspect of the present disclosure, a method includes receiving patient data corresponding to a TB patient. The patient data is associated with sputum time-to-positivity (TTP) data associated with a time period. The method further includes identifying, based on the patient data, a kill rate of semidormant/persistent (Yₛ) Mycobacterium tuberculosis. The method also includes determining a treatment response prediction result based on the kill rate and generating an output based on the treatment response prediction result.

### CROSS-REFERENCE TO RELATED APPLICATIONS

This application claims the benefit of priority to U.S. Provisional Patent Application Ser. No. 62/804,590, filed Feb. 12, 2019, hereby incorporated by reference in its entirety.

### TECHNICAL FIELD

The present disclosure generally relates to tuberculosis therapy (e.g., tuberculosis treatment), but not by way of limitation, to devices, systems, and methods to identify and use a TB biomarker for treating a patient with tuberculosis.

### STATEMENT REGARDING FEDERALLY SPONSORED RESEARCH

Not applicable.

### BACKGROUND OF THE INVENTION

Tuberculosis (TB) is among the top ten killers of mankind, and has killed over 1 billion people over the last century. Over the past few decades, efforts to develop new and shorter chemotherapy regimens have gathered steam. The standard approach has been to use a combination of newly discovered molecules with current anti-TB medications or repurposed antibiotics to build regimens for drug susceptible and drug resistant TB. With these approaches, it currently takes decades to build a new regimen and perform successful phase III clinical trials. One stumbling block to fast-track approval of these experimental regimens has been the lack of predictive biomarkers, as defined by BEST glossary, that have high sensitivity and specificity in predicting meaningful long-term outcomes such as relapse, microbiologic cure and failure. Eight week studies, and the 2-month sputum culture or smear status have been used for clinical trials; the 2-month sputum status is extensively used by TB programs worldwide but its sensitivity and specificity for predicting treatment failure and relapse is poor.

In early bactericidal activity (EBA) studies, serial sputum smears in the first two days of therapy are used as a common first step to examine the efficacy of new TB therapies by calculating the colony forming unit (CFU) kill rates (i.e., slopes). However, even when extended to 14 days (herein termed extended EBA rate) to capture some sterilizing effect, its accuracy in forecasting failure, relapse and cure remains insufficient. Subsequently, 8-week phase II studies, which rely on both time-to-conversion and the 2-month sputum status, are performed after the EBA studies. If a regimen performed well, then a large randomized controlled trial (Phase III) with long term outcomes such as relapse, death and cure, is performed. Such a study often lasts up to 5 years. Thus, a need exists to identify a clinically meaningful TB biomarker for early decision-making which may decrease the duration and cost of TB clinical trials while avoiding use of potentially catastrophic clinical endpoints such as therapy failure and death.

### SUMMARY

The present disclosure describes systems, devices, and methods for tuberculosis therapy (e.g., tuberculosis treatment). For example, the present disclosure describes a system in which patient data, such as time-to-positivity (TTP) values of sputum Mycobacterium tuberculosis (Mtb) liquid cultures over a period of treatment, such as the first eight weeks of treatment, is provided and analyzed to identify whether or not a patient is responding to treatment. In some implementations, the patient data (e.g., TTP values) are analyzed to calculate slopes and other variables that are predictors of response. To illustrate, non-linear ordinary differential equations (ODEs) are used to determine non-linear ODE kill rates, termed γ-slopes for different Mtb subpopulations: (i) fast-replicating (γ_{f}) and (ii) slow/non-replicating (γₛ). Additionally, or alternatively, the TTP values may be converted to a colony forming unit (CFU) count/mL, and a kill rate of semidormant/persistent (γₛ) Mycobacterium tuberculosis may be determined as log₁₀ CFU/ml/day for semidormant/persistent (γₛ) Mycobacterium tuberculosis. In some implementations, an initial bacterial burden (B(0)) is also determined based on the patient data. The system includes instructions (e.g., software including one or more algorithms) and data storage (e.g., a memory or database) that may be utilized by health clinics and TB programs which provide the patient data to the system via a computer, such as a laptop or smartphone. In turn, the system analyzes the patient data to determine treatment response prediction result which includes at least one of the following: (i) patient not responding, (ii) patient is responding, but will relapse, (iii) patient's trajectory means cure without relapse, (iv) patient is responding slowly and will need prolonged therapy duration. In addition to the treatment response prediction result (for 2, 4 and 6 months), the system may indicate a minimal duration of therapy required for cure (and avoid relapse), a shorter treatment duration, a dose increase of one or more anti-TB drugs, a switch of treatment regimens, or a longer treatment duration.

Thus, the present disclosure describes a computational and software platform to identify TB patients who are responding to therapy during a time period, such as during the first 8 weeks of therapy. Additionally, the present disclosure describes use of patient data (e.g., TTP values) as a biomarker for early decision-making during TB treatment. Such information enables health care professionals to decrease the duration and cost of TB clinical trials while avoiding use of potentially catastrophic clinical endpoints, such as therapy failure or death. For example, if a patient is identified during the first 8 weeks of therapy to have a therapy failure or a potential relapse, intervention can occur, such as dose increases or switching therapy regimens. As another example, slope determined during the analysis of the patient data can also be used to identify a patient who can be cured with shorter therapy duration.

Another aspect of the present disclosure describes systems, devices, and methods for tuberculosis therapy (e.g., tuberculosis treatment) which analyze and determine anti-TB drug dose(s). Illustrative, non-limiting examples of anti-TB drugs include isoniazid, rifampin, pyrazinamide, ethambutol, levofloxacin, gatifloxacin, amikacin, ethionamide, and cycloserine. The system described herein may receive the patient data that includes dosages of one or more anti-TB drugs received by a patient, concentrations of the one or more anti-TB drugs in the patient's body, and/or one or more clinical characteristics relevant to dosing, such as weight, biological sex, height, and TB site (e.g., lung, central nervous system, etc.) of the patient. The system is configured to compare the concentrations to one or more thresholds to determine if a concentration is toxic, if a dosage needs to be changed (e.g., increased or decreased), if a new dosage is recommended, and/or when to perform the next measurement of drug concentrations. The next measurement of drug concentrations may be performed to confirm that the required concentration thresholds have been achieved post dose change and/or that acceptable drug concentrations have been maintained.

Thus, the systems, devices, and methods described herein provide for monitoring and adjustment of dosages of one or more anti-TB drugs during TB treatment. Monitoring and adjustment of dosages of one or more anti-TB drugs during TB treatment advantageously enables avoidance of low drug concentrations despite initial and/or perceived “adequate” dosing. Additionally, adjustment of dosages of one or more anti-TB drugs during TB treatment beneficially decreases failure of therapy and development of drug resistance.

In an aspect of the present disclosure, a method for treating a patient with tuberculosis includes receiving patient data corresponding to a TB patient. The patient data is associated with sputum time-to-positivity (TTP) data associated with a time period. The method also includes identifying, based on the patient data, a kill rate of semidormant/persistent (γₛ) Mycobacterium tuberculosis. The method further includes determining a treatment response prediction result based on the kill rate and generating an output based on the treatment response prediction result.

In another aspect of the present disclosure, a system for treating a patient with tuberculosis includes a receiver configured to receive patient data corresponding to a TB patient. The patient data is associated with sputum time-to-positivity (TTP) data associated with a time period. The system further includes a processor coupled to the receiver and configured to: identify, based on the patient data, a kill rate of semidormant/persistent (γₛ) Mycobacterium tuberculosis; determine a treatment response prediction result based on the kill rate; and generate an output based on the treatment response prediction result.

In another aspect of the present disclosure, a non-transitory computer readable medium comprising instructions that, when executed by a processor, cause the processor to identify, based on patient data corresponding to a TB patient, a kill rate of semidormant/persistent (γₛ) Mycobacterium tuberculosis. The patient data is associated with sputum time-to-positivity (TTP) data associated with a time period. The instructions, when executed by the processor, further cause the processor to determine a treatment response prediction result based on the kill rate and generate an output based on the treatment response prediction result.

In another aspect of the present disclosure, a non-transitory computer readable medium comprising instructions that, when executed by a processor, cause the processor to identify, based on patient data corresponding to a TB patient, an initial bacterial burden (B(0)) associated with Mycobacterium tuberculosis. The initial bacterial burden (B(0)) is associated with a sputum time-to-positivity (TTP) value. The instructions, when executed by the processor, further cause the processor to determine a treatment response prediction result based on the initial bacterial burden (B(0)) and generate an output based on the treatment response prediction result.

In another aspect of the present disclosure, a method for treating a patient with tuberculosis includes receiving patient data corresponding to a TB patient; identifying, based on the patient data, a concentration of an anti-TB drug in a patient; performing a comparison between the concentration and one or more thresholds; and generating an output based on the comparison.

The foregoing has outlined rather broadly the features and technical advantages of the present disclosure in order that the detailed description that follows may be better understood. Additional features and advantages will be described hereinafter which form the subject of the claims. It should be appreciated by those skilled in the art that the conception and specific examples disclosed may be readily utilized as a basis for modifying or designing other structures for carrying out the same purposes described herein. It should also be realized by those skilled in the art that such equivalent constructions do not depart from the spirit and scope of the disclosure as set forth in the appended claims. The aspects which are characterized herein, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE FIGURES

For a more complete understanding of the present examples, reference is now made to the following descriptions taken in conjunction with the accompanying figures, in which:

FIG. 1 illustrates a block diagram of a system for use in treating tuberculosis;

FIG. 2 illustrates a flow diagraph of an example of a method of treating tuberculosis;

FIG. 3 illustrates a flow diagraph of an example of another method of treating tuberculosis;

FIG. 4 illustrates a flow diagraph of an example of another method of treating tuberculosis;

FIG. 5 is an illustration showing CFU/TTP trajectories of each individual patient of a cured patient cluster;

FIG. 6 is an illustration showing median log₁₀ CFU/mL plus interquartile range, for the follow up periods of 6 to 18 months, for the cured patient cluster;

FIG. 7 is an illustration showing CFU/TTP trajectories of each individual patient of a slow cure patient cluster;

FIG. 8 is an illustration showing box-plots of median log₁₀ CFU/mL plus interquartile range, for the follow up periods of 6 to 18 months, for the slow cure patient cluster after therapy completion;

FIG. 9 is an illustration showing CFU/TTP trajectories of each individual patient of a relapse patient cluster;

FIG. 10 is an illustration showing box-plots of median log₁₀ CFU/mL plus interquartile range, for the follow up periods of 6 to 18 months, for the relapse patient cluster after therapy completion;

FIG. 11 is an illustration showing CFU/TTP trajectories of each individual patient of a failed treatment patient cluster;

FIG. 12 is an illustration showing box-plots of median log₁₀ CFU/mL plus interquartile range, for the follow up periods of 6 to 18 months, for the failed treatment patient cluster after therapy completion;

FIG. 13 is a graph showing distributions of computed slopes γₛ (slow) at 2 months;

FIG. 14 is a graph showing distributions of computed slopes γₛ (slow) at 4 months;

FIG. 15 is a graph showing distributions of computed slopes γₛ (slow) at 6 months;

FIG. 16 is a graph showing distributions of computed slopes γ_{f} (fast) at 2 months;

FIG. 17 is a graph showing distributions of computed slopes γ_{f} (fast) at 4 months;

FIG. 18 is a graph showing distributions of computed slopes γ_{f} (fast) at 6 months;

FIG. 19 is an illustration showing pairwise statistical difference analysis for distributions of slopes at 2, 4 and 6 months between all the clusters (cure, slow-cure, relapse and failure) for the slow (γₛ) slopes illustrated with p-values;

FIG. 20 is an illustration showing pairwise statistical difference analysis for distributions of slopes at 2, 4 and 6 months between all the clusters (cure, slow-cure, relapse and failure) for the fast (γ_{f}) slopes illustrated with p-values;

FIG. 21 is a graph showing γₛ slopes to achieve cure within 6 months for patients with high bacterial burden compared to those with medium and lower bacterial burdens;

FIG. 22 is a graph showing γₛ slopes required to achieve cure at 2, 4 and 6 months or for delayed cure with +1 month up to +3 months are shown for a patient starting with high Mtb burdens;

FIG. 23 is a graph showing γₛ slopes required to achieve cure at 2, 4 and 6 months or for delayed cure with +1 month up to +3 months are shown for a patient starting with medium Mtb burdens;

FIG. 24 is a graph showing γₛ slopes required to achieve cure at 2, 4 and 6 months or for delayed cure with +1 month up to +3 months are shown for a patient starting with low Mtb burdens;

FIG. 25 is a graph showing Magnitudes of slopes for therapy duration of 1 and 3 months are extrapolated and interpolated in log₁₀ CFU/mL;

FIG. 26 is a graph showing Magnitudes of slopes for therapy duration of 1 and 3 months are extrapolated and interpolated TTP-slope;

FIG. 27 is a graph showing biomarker sensitivity, specificity and accuracy comparison of treatment failure vs. cure; and

FIG. 28 is a graph showing biomarker sensitivity, specificity and accuracy comparison of treatment failure vs. relapse.

### DETAILED DESCRIPTION OF THE INVENTION

Particular implementations of the present disclosure are described below with reference to the drawings. In the description, common features are designated by common reference numbers throughout the drawings. As used herein, various terminology is for the purpose of describing particular implementations only and is not intended to be limiting of implementations. For example, the singular forms “a,” “an,” and “the” are intended to include the plural forms as well, unless the context clearly indicates otherwise. It may be further understood that the terms “comprises” and “comprising” may be used interchangeably with “includes” or “including.” Additionally, it will be understood that the term “wherein” may be used interchangeably with “where.”

As used herein, an ordinal term (e.g., “first,” “second,” “third,” etc.) used to modify an element, such as a structure, a component, an operation, etc., does not by itself indicate any priority or order of the element with respect to another element, but rather merely distinguishes the element from another element having a same name (but for use of the ordinal term). The term “coupled” is defined as connected, although not necessarily directly, and not necessarily mechanically; two items that are “coupled” may be unitary with each other. The phrase “and/or” means and or. To illustrate, A, B, and/or C includes: A alone, B alone, C alone, a combination of A and B, a combination of A and C, a combination of B and C, or a combination of A, B, and C. In other words, “and/or” operates as an inclusive or.

Further, a device or system that is configured in a certain way is configured in at least that way, but it can also be configured in other ways than those specifically described. The feature or features of one embodiment may be applied to other embodiments, even though not described or illustrated, unless expressly prohibited by this disclosure or the nature of a described implementation.

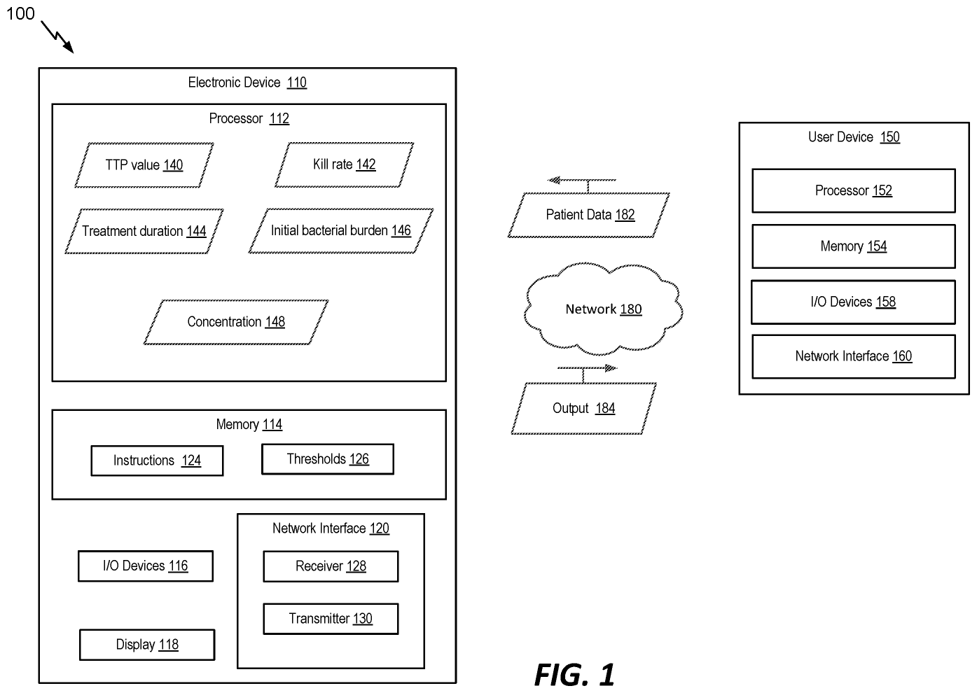

Referring to FIG. 1, a block diagram of an example of a system 100 for use in treating tuberculosis. For example, the system 100 may be used to determine and/or predict an effectiveness of treatment, determine whether to maintain or modify a treatment duration, and/or to determine whether or not to maintain or change a treat regimen and/or a drug dosage.

The system 100 includes an electronic device 110 and a user device 150. Electronic device 110 and user device 150 may be communicatively coupled via a network 180. Network 180 may include or correspond to a wireless network, a wired network, or a combination thereof. Electronic device 110 and/or user device 150 may include a smartphone, a tablet computing device, a personal computing device, a laptop computing device, a desktop computing device, a computer system, a server, etc. In some implementations, user device 150 is a mobile device (e.g., a smartphone) storing an application and electronic device 110 is a server that hosts and/or supports the application. Although system 100 is shown as having a single electronic device (e.g., 110) and a single user device (e.g., 150), system 100 is not to be limited. For example, system 100 may include multiple user devices (e.g., 150), such as a first user device associated with a patient and a second user device associated with a health care provider, clinic, and/or lab. To illustrate, the health care provider, clinic, and/or lab may be engaged in tuberculosis treatment of the patient.

Electronic device 110 includes a processor 112, a memory 114, one or more I/O device 116, a display 118, and a network interface 120. Processor 112 may be coupled to memory 114, one or more I/O device 116, display 118, and network interface 120. Processor 112 may include one or more processors, such as a baseband processor, an application processor, or both. For example, processor 112 may include a general purpose computer system (e.g., a personal computer (PC), a server, a tablet device, etc.) and/or a special purpose processor platform (e.g., application specific integrated circuit (ASIC), system on a chip (SoC), etc.).

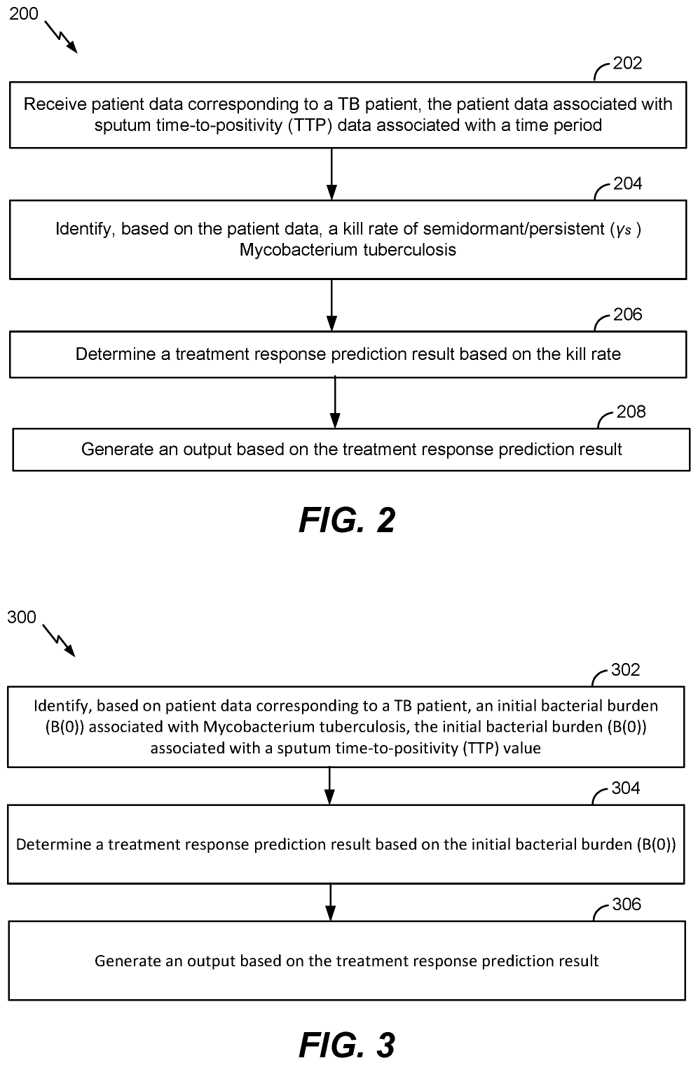

Memory 114 may include one or more memory devices, such as a RAM, a ROM, one or more HDDs, a flash memory, SSDs, other devices configured to store data in a persistent or non-persistent state, or a combination thereof, as illustrative, non-limiting examples. The memory 114 includes instructions 124 and one or more thresholds 126. In aspects, memory 114 may store the instructions 124 that, when executed by processor 112, cause the processor 112 to perform operations according to aspects of the present disclosure, as described herein, such as one or more operations as described with reference to at least FIGS. 2-4.

In some implementations, user device 150 receives patient data, such as patient data 182, which may be stored at memory 114. Additionally, or alternatively, memory 114 and/or a database (not shown) coupled and/or accessible to electronic device 110 may include historical patient data of a plurality of patients having tuberculosis. The one or more datasets which comprise the historical data of a plurality of patients having tuberculosis may be analyzed by executing a plurality of instructions via processor 112. For example, logistic regression may be performed on the one or more datasets to find a correlation between each of one or more variables and the patient's tuberculosis data. The correlation may be utilized to predict a treatment outcome, modify a treatment duration, modify a dose of an anti-tuberculosis medication, and/or identify a follow up date. To illustrate, the analysis of the historical data may include operations as described herein with reference to the example below having the heading “Bacterial load slopes as biomarkers of tuberculosis therapy success, failure and relapse.”

The one or more I/O devices 116 may include a mouse, a keyboard, a display device, a camera, other I/O devices, or a combination thereof. The network interface 120 may be configured to communicatively couple the electronic device 110 to one or more external devices, such as user device 150, via one or more networks (e.g., 180). For example, network interface 120 includes a receiver 128 and a transmitter 130.

In some implementations, electronic device 110 may be a single device configured to perform the operations described herein. Those of skill in the art would recognize that although FIG. 1 shows the electronic device 110 as a single block, the implementation of electronic device 110 is not limited to a single component, and instead may be distributed over several components. For example, operations of electronic device 110 may be distributed over multiple devices configured to perform all or a portion of the operations of the electronic device 110 in accordance with the present disclosure. Implementing electronic device 110 functionality over multiple devices may increase efficiency, processing time, and reliability of system 100.

User device 150 may be associated with and/or operated by a patient and/or a health care provider (such as a nurse, a physician, or the like) in a hospital ward, at the patient's home, or other places, to evaluate a patient's tuberculosis treatment. User device 150 includes one or more processors 152, a memory 154, one or more input/output (I/O) devices 158, and a network interface 160. Processor 152 may comprise various forms of processor-based systems in accordance with aspects described herein. For example, processor 152 may include a general purpose computer system (e.g., a personal computer (PC), a server, a tablet device, etc.) and/or a special purpose processor platform (e.g., application specific integrated circuit (ASIC), system on a chip (SoC), etc.).

Memory 154 may include ROM devices, RAM devices, one or more HDDs, flash memory devices, SSDs, other devices configured to store data in a persistent or non-persistent state, or a combination of different memory devices. In aspects, memory 154 may store the instructions that, when executed by processor 152, cause the processor 152 to perform operations. The instructions may be in the form of software, or software applications downloadable to user device 150. For example, a user may download the instructions from electronic device 110 through network 180. The instructions may enable processor 152 to perform operations at user device 150 and/or to communicate with electronic device 110.

The one or more I/O devices 158 may include a mouse, a keyboard, a display device, a camera, other I/O devices, or a combination thereof. The network interface 160 may be configured to communicatively couple the user device 150 to one or more external devices, such as electronic device 110.

In some implementations, user device 150 receives patient data, such as patient data 182, which may be stored at memory 154. The patient data may be associated with or correspond to one or more patients. The patient data (e.g., 182) may include information and/or measurements of a patient, such as the patients' respiratory rate, heart rate, diastolic blood pressure, age, quantitative/non-subjective mental status, pulse pressure, weight, biological sex, height, site of disease (lung, central nervous system, etc.), or other clinical and/or demographical information of the patient. Additionally, or alternatively, the patient data (e.g., 182) may include data and/or information that is associated with or corresponds to a sputum Mycobacterium tuberculosis (Mtb) liquid culture. For example, the patient data may include time-to-positivity (TTP) data (e.g., a TTP value), an Mtb kill rate (e.g., a first kill rate of a fast-replicating (γ_{f}) Mtb subpopulation, a second kill rate of a slow/non-replicating (γₛ) Mtb subpopulation, or both), time-to-extinction (TTE) data (e.g., a TTE value) of the entire bacterial population in the lungs, an elapsed therapy duration, an initial bacterial burden, or a combination thereof. In some implementations, the patient data 182 may additionally or alternatively include a list and/or an amount(s) of one or more drugs used in the patient's tuberculosis treatment, concentrations of the one or more drugs in the patient's system, or a combination thereof.

During operation, patient data 182 is communicated from user device 150 to electronic device 110. The patient data 182 is received by electronic device 110 and is stored in memory 114 and/or provided to processor 112. Based on patient data 182, processor 112 identifies and/or calculates one or more of a TTP value 140, a kill rate 142, a treatment duration 144, an initial bacterial burden 146, and a concentration 148. For example, TTP value 140, kill rate 142 (e.g., a slope), treatment duration 144, initial bacterial burden 146, and concentration 148 may be included in or indicated by patient data 182. In some implementations, processor 112 may calculate kill rate 142 or initial bacterial burden 146 as described further herein with reference to FIGS. 2-3. To illustrate, processor 112 may us non-linear ordinary differential equations (ODEs) to determine non-linear ODE kill rates, termed γ-slopes for different Mtb subpopulations: (i) fast-replicating (γ_{f}) and (ii) slow/non-replicating (γₛ), may be determined. Additionally, or alternatively, the TTP values may be converted to a colony forming unit (CFU) count/mL, and a kill rate of semidormant/persistent (γₛ) Mycobacterium tuberculosis may be determined as log₁₀ CFU/ml/day for semidormant/persistent (γₛ) Mycobacterium tuberculosis.

Processor 112 may compare at least one of TTP value 140, kill rate 142, initial bacterial burden 146, and concentration 148 to one or more thresholds 126. In some implementations, processor 112 may identify and access at least one of the one or more thresholds 126 based on treatment duration 144 (e.g., based on how long the patient has been undergoing TB treatment). Based on the comparison, processor 112 may generate output 184. Output 184 may be presented via I/O device 116, via display 118, or transmitted via network interface 120 (e.g., transmitter 130). For example, output 184, such as a message, display, indicator, etc., may be transmitted to user device 150 (which provided patient data 182), or to anther device.

In some implementations, processor 112 may analyze patient data 182 (e.g., TTP value 140, kill rate 142, and/or initial bacterial burden 146) to determine a treatment response prediction result which includes one of the following: (i) patient not responding, (ii) patient is responding, but will relapse, (iii) patient's trajectory means cure without relapse, (iv) patient is responding slowly and will need prolonged therapy duration. In addition to the treatment response prediction result, processor 112 may indicate a minimal duration of therapy required for cure (and avoid relapse), a shorter treatment duration, a dose increase of one or more anti-TB drugs, a switch of treatment regimen, or a longer treatment duration.

In some implementation, processor 112 may analyze patient data 182 (e.g., concentration 148) to determine anti-TB drug dose(s). To illustrate, processor 112 is configured to compare the concentrations to one or more thresholds to determine if a concentration is toxic, if a dosage needs to be changed (e.g., increased or decreased), if a new dosage is to be recommended, and/or when to perform the next measurement of drug concentrations. The next measurement of drug concentrations may be performed to confirm that the required concentration thresholds have been achieved post dose change and/or to confirm that one or more concentrations have been maintained at acceptable levels.

In some implementations, electronic device 110 executes an algorithm that includes calculations of slopes and other variables that are predictors of response, and intelligent machines based decision making and response. The algorithm is a learning algorithm and will continue machine learning of all uploaded/received data so that it gets more accurate as the database (e.g., stored patient data) grows. For example, the algorithm will continue machine learning of all uploaded data so that it gets more accurate in dose calculation as it grows.

Upon receiving output 184 from electronic device 110, user device 150 can display a representation of output 184 and/or trigger certain action(s) of treatment for the patient, for example, automatically notifying a nurse, a physician, a RRT, or other healthcare providers. In implementations, where system 100 includes multiple user devices, a first user device corresponding to a physician or lab can provide patient data 182 to electronic device 110 and electronic device can provide output 184 to the first user device, a second user device corresponding to the patient, or both.

In some implementations, operations described as being performed by electronic device 110 may be performed at user device 150. For example, user device 150 may include instructions 124 and thresholds 126. In such implementations, user device 150 may process patient data 182 and send output 184 to electronic device 110. Electronic device 110 receives output 184 and updates tracking data (e.g., a database), which may be used to update and/or refine one or more algorithms, one or more equations, one or more threshold (e.g., 126), etc. To illustrate, electronic device 110 may include machine learning to improve the one or more thresholds 126 as additional patient data (182), outputs (e.g., 184), or both are received and/or determined.

In a particular implementation, system 100 (e.g., electronic device 110) includes receiver 128 configured to receive patient data 182 corresponding to a TB patient. Patient data 182 may be associated with sputum time-to-positivity (TTP) data (e.g., 140) associated with a time period (e.g., 144). In such an implementation, system 100 (e.g., electronic device 110) further includes processor 112 coupled to receiver 128 and configured to identify, based on patient data 182, a kill rate of semidormant/persistent (γₛ) Mycobacterium tuberculosis (e.g., 142). Processor 112 is further configured to determine a treatment response prediction result based on the kill rate (e.g., 142) and generate output 184 based on the treatment response prediction result. In some such implementations, system 100 (e.g., electronic device 110) may further include display 118 (e.g., a display device) coupled to processor 112 and configured to provide a presentation based on output 184, and/or transmitter 130 configured to send output 184 (to user device 150). Additionally, or alternatively, system 100 (e.g., electronic device 110) may also include memory 114 coupled to processor 112 and configured to store at least one thresholds value (e.g., 126) and/or one or more instructions (e.g., 124) executable by processor 112 to perform one or more operations.

In some implementations of system 100, processor 112 may further be configured to identify treatment duration (e.g., 144) associated with patient data (e.g., 182), retrieve one or more threshold values (e.g., 126) from memory (e.g., 114) based on treatment duration (e.g., 144), and compare kill rate (e.g., 142) to the one or more threshold values (e.g., 126) to determine the treatment response prediction result. Additionally, or alternatively, processor 112 may be configured to determine initial bacterial burden (B(0)) (e.g., 146) based on patient data (e.g., 182), identify treatment duration (e.g., 144) associated with patient data (e.g., 182), retrieve one or more threshold values (e.g., 126) from memory (e.g., 114) base on treatment duration (e.g., 144), and compare initial bacterial burden (B(0)) (e.g., 146) to one or more threshold values (e.g., 126) to determine the treatment response prediction result. In a particular implementation, processor 112 is configured to identify TTP value (e.g., 140) based on patient data (e.g., 182), calculate initial bacterial burden (B(0)) (e.g., 146) based on TTP value (e.g., 140) such that the treatment response prediction result is further determined based on the initial bacterial burden (B(0) (e.g., 146).

In a particular implementation, a non-transitory computer readable medium (e.g., 114) comprising instructions (e.g., 124) that, when executed by processor 112, cause processor 112 to identify, based on patient data 182 corresponding to a TB patient, a kill rate of semidormant/persistent (γₛ) Mycobacterium tuberculosis (e.g., 142). The patient data 182 is associated with sputum time-to-positivity (TTP) data associated with a time period (e.g., 144). The instructions (e.g., 124), when executed by processor 112, further cause processor 112 to determine a treatment response prediction result based on the kill rate (e.g., 142) and generate output 184 based on the treatment response prediction result.

In another particular implementation, a non-transitory computer readable medium (e.g., 114) comprising instructions (e.g., 124) that, when executed by processor 112, cause processor 112 to identify, based on patient data 182 corresponding to a TB patient, an initial bacterial burden (B(0)) (e.g., 146) associated with Mycobacterium tuberculosis. The initial bacterial burden (B(0)) is associated with a sputum time-to-positivity (TTP) value. The instructions (e.g., 124), when executed by processor 112, further cause processor 112 to determine a treatment response prediction result based on initial bacterial burden (B(0)) (e.g., 146) and generate output 184 based on the treatment response prediction result.

Accordingly, system 100 advantageously provides a computational and software platform to identify TB patients who are responding to therapy during a time period, such as during the first 8 weeks of therapy. Use of patient data 182 (e.g., TTP values) for use as a biomarker for early decision-making during TB treatment is an efficient way to analyze long-term treatment outcomes because such data is already acquired in conventional clinical trials and TB treatment and available for analysis. The predicted effectiveness of a particular TB treatment enables health care professionals to decrease the duration and cost of TB clinical trials while avoiding use of potentially catastrophic clinical endpoints such as therapy failure and death. For example, if a patient is identified/predicted during the first 8 weeks of therapy to have a therapy failure or a potential relapse, intervention can occur, such as dose increases or switching therapy regimens. As another example, slope determined during the analysis of the patient data can also be used to identify a patient who can be cured with shorter therapy duration.

Referring to FIGS. 2-4, methods of treating tuberculosis are shown. For example, each of the methods of FIG. 2-4 may be performed by system 100, such as electronic device 110 (e.g., processor 112) and/or user device 150 (e.g., processor 152).

Referring to FIG. 2, a method 200 of treating tuberculosis includes receiving patient data corresponding to a TB patient, the patient data associated with sputum time-to-positivity (TTP) data associated with a time period, at 202. The patient data may include or correspond to patient data 182. For example, the patient data includes a TTP value, a colony forming unit (CFU) count/mL, a kill rate of the semidormant/persistent (γₛ) Mycobacterium tuberculosis, an initial bacterial burden, or a combination thereof. The time period may include or correspond to a treatment duration (e.g., an elapsed amount of time that the patient has been treated), such as treatment duration 144. For example, the time period may be less than, greater than, equal to, and/or approximately two months (e.g., eight weeks), four months, or six months, as illustrative, non-limiting examples.

In some implementations, the patient data includes a TTP value, a colony forming unit (CFU) count/mL, a kill rate of the semidormant/persistent (γₛ) Mycobacterium tuberculosis, an initial bacterial burden, or a combination thereof. Additionally, or alternatively, the patient data may include information and/or measurements of a patient, such as the patients' respiratory rate, heart rate, diastolic blood pressure, age, quantitative/non-subjective mental status, pulse pressure, weight, biological sex, height, site of disease (lung, central nervous system, etc.), or other clinical and/or demographical information of the patients. Additionally, or alternatively, the patient data may include data and/or information that is associated with or corresponds to a sputum Mycobacterium tuberculosis (Mtb) liquid culture. For example, the patient data may include time-to-positivity (TTP) data (e.g., a TTP value), an Mtb kill rate (e.g., a first kill rate of a fast-replicating (γ_{f}) Mtb subpopulation, a second kill rate of a slow/non-replicating (γₛ) Mtb subpopulation, or both), time-to-extinction (TTE) data (e.g., a TTE value) of the entire bacterial population in the lungs, an elapsed therapy duration, an initial bacterial burden, or a combination thereof. In some implementations, the patient data may additionally or alternatively include a list and/or an amount(s) of one or more drugs used in the patient's tuberculosis treatment, concentrations of the one or more drugs in the patient's system, or a combination thereof.

The method 200 also includes identifying, based on the patient data, a kill rate of semidormant/persistent (γₛ) Mycobacterium tuberculosis, at 204. For example, the kill rate may include or correspond to kill rate 142. In some implementations, method 200 may include calculating the kill rate of the semidormant/persistent (γₛ) Mycobacterium tuberculosis based on the patient data. For example, calculating the kill rate of the semidormant/persistent (γₛ) Mycobacterium tuberculosis may include converting TTP data (e.g., TTP value 140) to a colony forming unit (CFU) count/mL and determining the kill rate as log₁₀ CFU/ml/day for semidormant/persistent (γₛ) Mycobacterium tuberculosis.

The method 200 also includes determining a treatment response prediction result based on the kill rate, at 206. The treatment response prediction result can be determined from the group consisting of: treatment failure, cure—but will relapse, cure without relapse, and slow-cure. The method 200 also includes generating an output based on the treatment response prediction result, at 208. For example, the output may include or correspond to output 184. In a particular implementation, the output indicates the treatment response prediction result.

In some implementations, the method 200 may further include determining a treatment recommendation based on the treatment predication result. In some such implementations, the output indicates the treatment recommendation. For example, when the treatment response prediction result is cure without relapse, the treatment recommendation indicates a shorter treatment duration. As another example, when the treatment response prediction result is treatment failure, the treatment recommendation indicates dose increase of one or more anti-TB drugs. As another example, when the treatment response prediction result is treatment failure, the treatment recommendation indicates to switch a treatment regimen. Additionally, when the treatment response prediction result is cure—but will relapse, the treatment recommendation indicates a longer treatment duration. As another example, when the treatment response prediction result is cure—but will relapse, the treatment recommendation indicates dose increase of one or more anti-TB drugs. Additionally, when the treatment response prediction result is cure—but will relapse, the treatment recommendation indicates to switch a treatment regimen.

In some implementations, when the patient data corresponds to a time period of 2 months of treatment, method 200 further includes determining, based on the patient data, whether the kill rate of the semidormant/persistent (γₛ) Mycobacterium tuberculosis is greater than or equal to 0.15. In such implementations, the treatment response prediction result may include cure without relapse in response to a determination that the kill rate of the semidormant/persistent (γₛ) Mycobacterium tuberculosis is greater than or equal to 0.15. Additionally, or alternatively, when the patient data corresponds to a time period of 4 months of treatment, method 200 may further include determining, based on the patient data, whether the kill rate of the semidormant/persistent (γₛ) Mycobacterium tuberculosis is greater than or equal to 0.14. In such implementations, the treatment response prediction result may include cure without relapse in response to a determination that the kill rate of the semidormant/persistent (γₛ) Mycobacterium tuberculosis is greater than or equal to 0.14. Additionally, or alternatively, when the patient data corresponds to a time period of 6 months of treatment, method 200 may further include determining, based on the patient data, whether the kill rate of the semidormant/persistent (γₛ) Mycobacterium tuberculosis is greater than or equal to 0.11. In such implementations, the treatment response prediction result may include cure without relapse in response to a determination that the kill rate of the semidormant/persistent (γₛ) Mycobacterium tuberculosis is greater than or equal to 0.11

In some implementations, when the patient data corresponds to a time period of 2 or 4 months of treatment, method 200 may further include determining, based on the patient data, whether the kill rate of the semidormant/persistent (γₛ) Mycobacterium tuberculosis is less than or equal to 0.1. In some such implementations, the treatment response prediction result may include treatment failure in response to a determination that the kill rate of the semidormant/persistent (γₛ) Mycobacterium tuberculosis is less than or equal to 0.1.

In some implementations, when the patient data corresponds to a time period of 2 months of treatment, method 200 may further include determining, based on the patient data, whether the kill rate of the semidormant/persistent (γₛ) Mycobacterium tuberculosis is greater than or equal to 0.1 and less than or equal to 0.15, and determining, based on the patient data, whether an initial bacterial burden (B(0)) is greater than or equal to 4.5 log₁₀ CFU/mL (TTP=8.11 days). In some such implementations, the treatment response prediction result may include cure—but will relapse in response to a determination that the kill rate of the semidormant/persistent (γₛ) Mycobacterium tuberculosis is between 0.1 and 0.15 and a determination that the initial bacterial burden (B(0)) is greater than or equal to 4.5 log₁₀ CFU/mL (TTP=8.11 days).

In some implementations, when the patient data corresponds to a time period of 4 months of treatment, method 200 may further include determining, based on the patient data, whether the kill rate of the semidormant/persistent (γₛ) Mycobacterium tuberculosis is greater than or equal to 0.09 and less than or equal to 0.14, and determining, based on the patient data, whether an initial bacterial burden (B(0)) is greater than or equal to 5.4 log₁₀ CFU/mL (TTP=5.93 days). In some such implementations, the treatment response prediction result may include cure—but will relapse in response to a determination that the kill rate of the semidormant/persistent (γₛ) Mycobacterium tuberculosis is between 0.09 and 0.14 and a determination that the initial bacterial burden (B(0)) is greater than or equal to 5.4 log₁₀ CFU/mL (TTP=5.93 days).

In some implementations, when the patient data corresponds to a time period of 2 or 6 months of treatment, method 200 may further include determining, based on the patient data, whether the kill rate of the semidormant/persistent (γₛ) Mycobacterium tuberculosis is greater than or equal to 0.1 and less than or equal to 0.15, and determining, based on the patient data, whether an initial bacterial burden (B(0)) is greater than or equal to 5.6 log₁₀ CFU/mL (TTP=5.49 days). In some such implementations, the treatment response prediction result may include cure—but will relapse in response to a determination that the kill rate of the semidormant/persistent (γₛ) Mycobacterium tuberculosis is between 0.1 and 0.15 and a determination that the initial bacterial burden (B(0)) is greater than or equal to 5.6 log₁₀ CFU/mL (TTP=5.49 days).

In some implementations, the method 200 may determine the treatment response prediction result based one or more comparisons as described herein with reference to the example below having the heading “Bacterial load slopes as biomarkers of tuberculosis therapy success, failure and relapse.” For example, the one or more comparisons may be associated with or correspond to Table 2 of the example below having the heading “Bacterial load slopes as biomarkers of tuberculosis therapy success, failure and relapse.” Additionally, machine learning of system 100 (e.g., electronic device 110) may employ a similar or the same process/methodology as described with reference to the example below having the heading “Bacterial load slopes as biomarkers of tuberculosis therapy success, failure and relapse” to determine and generate updated thresholds (e.g., 126) that are stored at memory 114 and utilized by processor 112.

Referring to FIG. 3, a method 300 of treating tuberculosis includes identifying, based on patient data corresponding to a TB patient, an initial bacterial burden (B(0)) associated with Mycobacterium tuberculosis, the initial bacterial burden (B(0)) associated with a sputum time-to-positivity (TTP) value, at 302. For example, the patient data may include or correspond to patient data 182. Additionally, the initial bacterial burden (B(0)) may include or correspond to initial bacterial burden 146.

The method 300 also includes determining a treatment response prediction result based on the initial bacterial burden (B(0)), at 304. The treatment response prediction result is determined as one of: treatment failure, cure—but will relapse, cure without relapse, and slow-cure. The method 300 also includes generating an output based on the treatment response prediction result, at 306. For example, the output may include or correspond to output 184.

Accordingly, each of methods 200 and 300 may advantageously use patient data (e.g., TTP values) as a biomarker for early decision-making during TB treatment. For example, the biomarker is used to determine treatment response prediction result which includes at least one of the following: (i) patient not responding, (ii) patient is responding, but will relapse, (iii) patient's trajectory means cure without relapse, (iv) patient is responding slowly and will need prolonged therapy duration. In addition to the treatment response prediction result, the system may indicate the minimal duration of therapy required for cure (and avoid relapse), a shorter treatment duration, dose increase of one or more anti-TB drugs, to switch a treatment regimen, or a longer treatment duration. For example, if a patient is identified during the first 8 weeks of therapy to have a therapy failure or a potential relapse, intervention can occur, such as dose increases or switching therapy regimens.

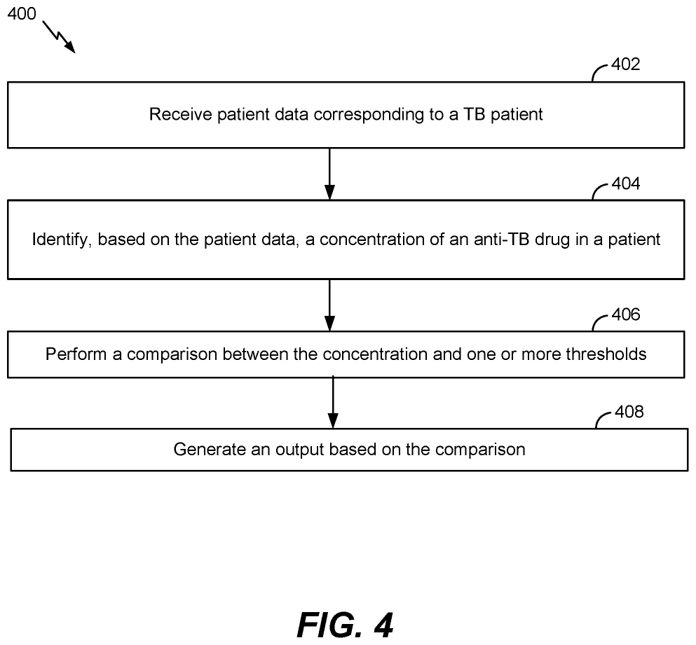

Referring to FIG. 4, a method 400 of treating tuberculosis includes receiving patient data corresponding to a TB patient, at 402. For example, the patient data may include or correspond to patient data 182. The method 400 also includes identifying, based on the patient data, a concentration of an anti-TB drug in a patient, at 404. For example, the concentration may include or correspond to concentration 148. The anti-TB drug includes isoniazid, rifampin, pyrazinamide, ethambutol, levofloxacin, gatifloxacin, amikacin, ethionamide, or cycloserine. The method 400 also includes performing a comparison between the concentration and one or more thresholds, at 406. The one or more thresholds may include or correspond to thresholds 126.

The method 400 further includes generating an output based on the comparison, at 408. For example, the output may include or correspond to output 184. The output may indicate to increase a dose of the anti-TB drug, an amount to adjust the dose of the anti-TB drug, a toxicity condition associated with the concentration of the anti-TB drug in the patient, a time to perform a next measurement of drug concentration, or a combination thereof. To illustrate, the output may indicate to reduce a dose of the anti-TB drug based on the toxicity condition.

In some implementations, method 400 further includes identifying a current dose that patient is receiving. The current does may be included in or indicated by the patient data (e.g., 182). Additionally, or alternatively, method 400 includes identifying at least one clinical characteristics associated with the patient. For example, the at least one clinical characteristic may include weight, biological sex, height, and/or tuberculosis site of the patient, as illustrative, non-limiting examples.

Accordingly, method 400 may advantageously enables monitoring and adjustment of dosages of one or more anti-TB drugs during TB treatment. Monitoring and adjustment of dosages of one or more anti-TB drugs during TB treatment advantageously enables avoidance of low drug concentrations despite initial and/or perceived “adequate” dosing. Additionally, adjustment of dosages of one or more anti-TB drugs during TB treatment beneficially decrease failure of therapy and development of drug resistance.

Thus, each of method 200 of FIG. 2, method 300 of FIG. 3, and method 400 of FIG. 4 enables treatment of tuberculosis. Methods 200, 300, 400 can be combined such that one or more operations described with reference to one of the methods of FIGS. 2-4 may be combined with one or more operations of another of FIGS. 2-4. For example, one or more operations of method 300 may be combined with one or more operations of method 400. Additionally, or alternatively, one or more operations described above with reference to FIG. 1 may be combined with one or more operations of one of FIGS. 2-4, or of a combination of FIGS. 2-4.

### Example(s)

The following describe scenarios that may be used with various embodiments of the present disclosure. These examples are not intended to be limiting, but rather to provide specific uses for different embodiments described herein.

### Bacterial Load Slopes as Biomarkers of Tuberculosis Therapy Success, Failure and Relapse

### A. Background

Tuberculosis clinical trials and routine tuberculosis programs require follow up of several years to document long-term outcomes such as relapse.

Tuberculosis (TB) is among the top ten killers of mankind, and has killed over 1 billion people over the last century. Over the past few decades, efforts to develop new and shorter chemotherapy regimens have gathered steam. (See Dheda K et al. (2017); Gillespie S H et al. (2014); Rosenthal I M et al. (2007); and Van D A et al. (2010)). The standard approach has been to use a combination of newly discovered molecules with current anti-TB medications or repurposed antibiotics to build regimens for drug susceptible and drug resistant TB. (See Tasneen R et al. (2014); Deshpande D et al. (2018); and Deshpande D et al. (2017)). With these approaches, it currently takes decades to build a new regimen and perform successful phase III clinical trials. One stumbling block to fast-track approval of these experimental regimens has been the lack of predictive biomarkers, as defined by BEST glossary, that have high sensitivity and specificity in predicting meaningful long-term outcomes such as relapse, microbiologic cure and failure. (See BEST (2016); and Burman W J (2003)). Eight week studies, and the 2-month sputum culture or smear status have been used for clinical trials; the 2-month sputum status is extensively used by TB programs worldwide but its sensitivity and specificity for predicting treatment failure and relapse is poor. (See Home D J et al. (2010); and Wallis R S et al. (2013)). Recently, sputum smear at start of therapy was shown to identify patients likely to benefit from shorter duration therapy. (See Imperial M Z et al. (2018)).

In early bactericidal activity (EBA) studies, serial sputum smears in the first two days of therapy are used as a common first step to examine the efficacy of new TB therapies by calculating the colony forming unit (CFU) kill rates (i.e., slopes). However, even when extended to 14 days (herein termed extended EBA rate) to capture some sterilizing effect, its accuracy in forecasting failure, relapse and cure remains paltry and controversial. (See Burman W J (2003); Jindani A et al. (1980); Jindani A et al. (2003); and Diacon A H et al. (2012)). Subsequently, 8-week phase II studies, which rely on both time-to-conversion and the 2-month sputum status, are performed after the EBA studies. (See Conde M B et al. (2009)). If a regimen performed well, then a large randomized controlled trial [Phase III] with long term outcomes such as relapse, death and cure, is performed. Such a study often lasts up to 5 years. This is similar to the constraints to fast tracking HIV regimens in the 1990s. Identification of viral load as a surrogate of efficacy in 1995 dramatically cut the duration and costs of HIV clinical trials, while avoiding use of potentially catastrophic clinical endpoints such as therapy failure and death. (See Mellors J W et al. (1996)). Here, we sought to identify clinically meaningful TB biomarkers for early decision-making that could be used in a similar fashion. Specifically, we were interested in a biomarker early during TB therapy that could predict clinical outcomes at the end of therapy and identify those patients who would fail or relapse after a long-term follow-up of up to 2 years. (See Mellors J W et al. (1996)).

Time-to-positivity (TTP) of sputum Mycobacterium tuberculosis (Mtb) liquid cultures at time of diagnosis correlate with long-term outcomes in TB patients. (See Epstein M D et al. (1998); Hesseling A C et al. (2010); and Pasipanodya J G et al. (2015)). We have found a high correlation between TTP in sputum and lung cavities. (See Dheda et al. (2018)). We recently used sputum TTP from two separate TB clinical trials to develop a set of non-linear ordinary differential equations (ODEs) that identifies both Mtb kill rates and the time-to-extinction (TTE) of the entire bacterial population in the lungs. (See Magombedze G et al. (2018)). We distinguished the non-linear ODE kill rates, which we termed γ-slopes for different Mtb subpopulations: (i) fast-replicating (γ_{f}) and (ii) slow/non-replicating (γₛ). (See Jindani A et al. (2003); and Gillespie S H et al. (2002)). We used these trajectories to calculate TTE values that also mark the minimal duration of therapy required for cure (and avoid relapse): all therapy failure and relapse is because of failure to eradicate the entire bacterial population. Here, we utilized TTP-based γ slopes to identify magnitudes of γ slopes predictive of long-term outcomes in patients on therapy for 2, 4 and 6 months, and then determined the accuracy and sensitivities of these biomarkers.

### B. Methods

We partitioned the ReMOX clinical trial study (N=1,931) data into two: derivation and validation datasets. We employed a computational framework using sputum time-to-culture-positivity (TTP) to derive kill rates (γ-slope) for rapidly multiplying (γ_{f}) and semidormant/persistent (γₛ) Mycobacterium tuberculosis and calculated time-to-extinction of the lung bacterial populations in the derivation dataset. Sensitivities and specificities, with 95% credible intervals [CI], of the derived biomarkers were calculated in the validation datasets for different therapy durations.

1. Study Design

The study design used data from the Rapid Evaluation of Moxifloxacin in Tuberculosis-TB (ReMOX-TB) study, a phase III clinical trial conducted at 50 sites across the world, and reported in full three years ago. (See Gillespie S H et al. (2014)). The data was mapped and curated by the Critical Path to TB Drug Regimes (CPTR). Patients with bacteriologically confirmed TB, based on at least two minimum sputum culture positive results, were enrolled in the ReMOX-TB study. (See Gillespie S H et al. (2014)). Patients' sputum was cultured in the Mycobacteria Growth Indicator Tube (MGIT). Patients were enrolled for our analyses regardless of initial drug susceptibility test or compliance with therapy, since the aim was to develop a method agnostic of regimen used and drug-resistance patterns. However, patients with majority of sputum samples that were contaminated, or missing or described as invalid results were excluded.

2. Data Extraction and Definition of Terms

Patient demographics (age, gender, race), clinical data (therapy regimens, serial TTP, serial colony forming unit (CFU) counts) and trial sites information (country) data was extracted from the CPTR website (http://www.cptrinitiative.org). First, in the derivation stage, outcomes definitions took into account the calculated time-to-extinction (TTE) of bacterial population, defined as complete depletion of all bacteria subpopulations in lungs of TB patients, which were set at 10⁻² colonies (3-fold lower than the MGIT assay lower limit of detection which corresponds to 0.24 log₁₀ CFU/mL). (See Magombedze G et al. (2018)). Microbiologic cure was defined as two negative sputum cultures without an intervening positive using MGIT TTP readout. Relapse was defined by the re-appearance of a positive smear in patients deemed cured at the end of therapy. Failure was defined as patients who failed to attain microbiologic cure at the end of therapy, as in the ReMOX-TB study protocol. These definitions are consistent with those currently employed by the World Health Organization (WHO) in evaluating program.

3. Data Partitioning

Patients in the standard TB therapy regimen (controls) were randomly partitioned into two subsets of equal size. The first set was designated as the model derivation set, while the remainder was assigned for use in model validation (validation data set). To capture sufficient relapse events, only patients with at least two consecutive sputum samples during follow-up after treatment were used in model training and cross validation. Patients who received the experimental ReMOX-TB arms (isoniazid or ethambutol arm) were used only in the validation dataset for sensitivity and specificity of predictors with 4 months therapy duration.

4. Mathematical Modeling of Bacteria Subpopulations

First, TTPs were converted to CFU/mL. (See Magombedze G et al. (2018)). Next, a model was developed that included one or more equations. (See Magombedze G et al. (2018)). The model gave us the ability to identify trajectories of bacterial subpopulations in time for each patient, and the TTE as well as corresponding kill rates or slopes log₁₀ CFU/ml/day for fast-replicating (γ_{f}) and semidormant/persistent (γₛ) Mtb. This enabled computing the proportion of trajectories (and thus patients) that did not achieved Mtb subpopulation extinction for 2, 4, or 6 months of TB therapy, and are thus likely to fail therapy.

5. Classification and Regression Tree Analysis to Rank Predictors of Outcome

Classification regression trees (CART) analyses is an agnostic machine learning method that has a classifier function, which has previously been used to predictors of TB outcomes. (See Swaminathan S et al. (2016); and Pasipanodya J G et al. (2013)). Clinical and radiological factors were examined, as well as model derived γₛ and γᵣ slopes and the initial bacterial burdens (B(0)), as potential predictors of outcome, defined as either therapy success at end of therapy, therapy failure (failure at the end of treatment or relapse). Such steps have been described in detail in the past. (See Swaminathan S et al. (2016); and Pasipanodya J G et al. (2013)). CART was implemented in Salford Miner.

6. Clustering and Model to Data Fitting

TTP trajectories were clustered using the K-means algorithm implemented in the KML-package in R. (See Genolini C et al. (2011)). The resulting clusters were the cure, slow-cure, relapse and failure clinical outcomes defined above. The subgroup of ‘slow cures’ were defined to denote those patients who had delayed attainment of microbiologic cure, but eventually got cured. First, the 6-month therapy data for each cluster was reduced to derive (i) the 4-month slopes (using the first 4 months accrued data) and (ii) the 2-month slopes (based on the first 2 months accrued data). Then, the model was fitted to data for each separate cluster and their respective reduced subsets. Model fitting was carried out identically for data in the different clusters. The Markov chain Monte Carlo (MCMC) method in R was implemented to estimate the drug kill parameters using 50, 000 runs of the chain. (See Magombedze G et al. (2018); Magombedze G et al. (2017); and Soetaert K et al. (2010).

7. Estimating and Evaluating Biomarkers at 2, 4 and 6 Months Using Three Methods

Estimates for therapy γₛ-slopes and γᵣ-slopes from model fitting for each treatment cluster were analyzed. The magnitudes of these estimates were explored as potential biomarkers for treatment outcomes at 2, 4, and 6 months using three independent methods. First, model fitting was used to determine slopes that characterize each specific cluster of treatment outcomes. Second, CART, implemented both in R (Rpart), was used with the model derived γₛ and γᵣ slopes and the initial bacterial burdens (B(0)) to identify threshold values via its regression function. Third, the derivation dataset parameters was used in domain of input for simulations in order to identify biomarker thresholds and delineate the tradeoff between the γ-slopes and the initial bacteria burden in predicting cure, relapse and treatment failure, at the boundaries of slopes that predicted outcomes.

8. Sensitivity Analysis for Treatment Duration

Simulations were carried out to determine the changes in γₛ values that resulted in treatment duration shortening (at 2 and 4 months) and those that led to prolonged treatment (7, 8 and 9 months). Magnitudes that correspond to these treatment end-points were determined relative to different categories of patient initial bacterial load, (i) high (5-6.5 log₁₀ CFU/mL), (ii) medium (3.5-5 log₁₀ CFU/mL) and (iii) low (less than 3.5 log₁₀ CFU/mL). These bounds were selected to toggle between CART discrete bounds and sweep across continuous patient CFU burdens for a wider scope on slope magnitudes.

9. Statistical Analysis

Mean values between groups were compared using Student's t-test or analysis of variance (ANOVA) F-test, while the Mann-Whitney test was used for proportions. Both the Spearman's correlations and un-weighted Cohen' Kappa coefficients were used to examine correlation and agreement, respectively, of clinical outcomes derived from ReMOX-TB study definition versus those derived from the model based on TTE. All analyses were performed with packages in R.

10. Validation of Identified Biomarkers

Individual patient TTP trajectories were fitted to the model to identify the corresponding γₛ, γ_{f} and TTE estimates in the validation datasets. The accuracy, sensitivity, and specificity of the derived markers to predict patients who were cured, relapsed, and failed therapy for the 6 months duration of therapy were calculated using the validation data set for standard therapy. In order to validate the first 8-week derived biomarkers for predicting therapy outcomes at end of four months therapy duration, we used the two ReMOX TB experimental arms (isoniazid or ethambutol).

### C. Results

Initial TTP>5.08 days and first 8 weeks-derived γₛ≤3.91 best predicted 6-month therapy duration outcome (AUC=0.88) in derivation dataset; in validation dataset sensitivities and specificities were 92 (CI: 78-98)%, and 86 (CI: 80-98)% for predicting failure-versus-cure and 92 (CI: 78-98)% and 89 (CI: 67-98)% for therapy failure-versus-relapse, respectively. For context, the sensitivities and specificities of the 2-month sputum smear/cultures were 33 (17-53)% and 71 (64-78)% for cure, respectfully. For the 4-months therapy duration, initial TTP>5.93 days and γₛ≤3.91 were predictive of therapy outcome (AUC=0.98) in the derivation dataset. In validation datasets, the sensitivities and specificities for relapse and failure were either 81 (CI: 70-90)% and 87 (CI: 83-90)% or 70 (CI: 60-79)% and 71 (CI: 67-75)% in the two 4-months duration experimental therapy arms. Initial TTP>6.15 days and γₛ≤5.99 best predicted 2-month therapy duration outcome (AUC=0.98) in derivation dataset.

1. Clinical, Microbial and Demographic Comparisons of Patients

Table 1 shows 1924 (99.8%) patients out of 1931 enrolled; we excluded 7 patients who had insufficient sputum samples. There were 637 (33%) patients in standard therapy arm, and 654 (34%) in the isoniazid arm and 633 (33%) in the ethambutol arm. The derivation dataset comprised 318 (50%) patients on standard therapy, and the validation datasets 319 patients on standard therapy for 6 months duration of therapy validation and the 1,287 patients in the experimental arms for 4-month therapy duration. Demographic and clinical characteristics were virtually identical between the derivation data sets and validation data sets in the standard treatment arms.

TABLE 1
Clinical features in 1,924 patients in derivation and validation datasets

| | Total | | | | |
|---|---|---|---|---|---|
| | sample | Experimental regimen arm | | Standard arm | |
| | N = 1924 | Ethambutol; | Isoniazid; | Training | Validation |
| Variable | (%) | n = 633 (%) | n = 654 (%) | n = 318 (%) | n = 319 (%) |
| Age, years | 33.40 (12.16) | 33.88 (12.15) | 32.89 (12.17) | 33.10 (11.93) | 33.81 (12.40) |
| [mean (SD)] | | | | | |
| Sex, Female | 585 (30) | 188 (30) | 205 (31) | 91 (29) | 101 (32) |
| Sex, Male | 1339 (70) | 223 (70) | 449 (69) | 227 (71) | 218 (68) |
| Race | | | | | |
| Black | 861 (45) | 289 (46) | 277 (42) | 149 (47) | 146 (46) |
| Asian | 586 (30) | 193 (30) | 201 (31) | 96 (30) | 96 (30) |
| Mixed | 451 (23) | 142 (22) | 169 (26) | 66 (21) | 74 (23) |
| Other | 26 (1) | 9 (1) | 7 (1) | 7 (2) | 3 (1) |
| Country site | | | | | |
| China | 22(1) | 5 (1) | 9 (1) | 6 (2) | 2 (1) |
| India | 372 (19) | 126 (20) | 127 (19) | 58 (18) | 61 (19) |
| Kenya | 136 (7) | 43 (7) | 49 (7) | 26 (8) | 18 (6) |
| Mexico | 22 (1) | 8 (1) | 5 (1) | 7 (2) | 2 (1) |
| Malaysia | 69 (4) | 20 (3) | 26 (4) | 10 (3) | 13 (4) |
| Thailand | 119 (6) | 41 (6) | 38 (6) | 21 (7) | 19 (6) |
| Tanzania | 211 (11) | 73 (12) | 64 (10) | 37 (12) | 37 (12) |
| South Africa | 908 (47) | 297 (47) | 313 (48) | 142 (45) | 156 (49) |
| Zambia | 65 (3) | 20 (3) | 23 (4) | 11 (3) | 11 (3) |

2. Correlation Between TTE-Based with Clinical Trial Definitions of Outcome

The number of patients deemed cured at different time intervals in the course of treatment obtained by counting the number of negative smears versus those using TTE model-based trajectories definitions had a Spearman rank correlation of 1 (p=0.0167). Other rank correlation methods (Kendall and Pearson) also demonstrated strong correlations (p=0.05). This means that the TTE and γ slope-based definitions were highly concordant with standard clinical definitions of outcome (Cohen's kappa of 0.65, p<0.0001). The Spearman rank correlation between γ_{f} (slope for log-phase growth Mtb) and extended EBA was 0.68 (p<0.0001), which suggests that the extended EBA mainly measures effect on log-phase growth Mtb.

3. CART Analysis to Rank Predictors of Outcome

In the derivation dataset of 318 patients, CART analysis identified the sterilizing slopes (γₛ) [variable importance score 100%] was the primary predictor followed by initial TTP (score 91.7%). γ_{f} was not chosen as a predictor. This post-test validation AUC was >85%, demonstrating how well initial TTP and γₛ will perform as predictors in a future dataset.

4. Biomarker Characterization Using Clustered Homogeneous Treatment Outcomes

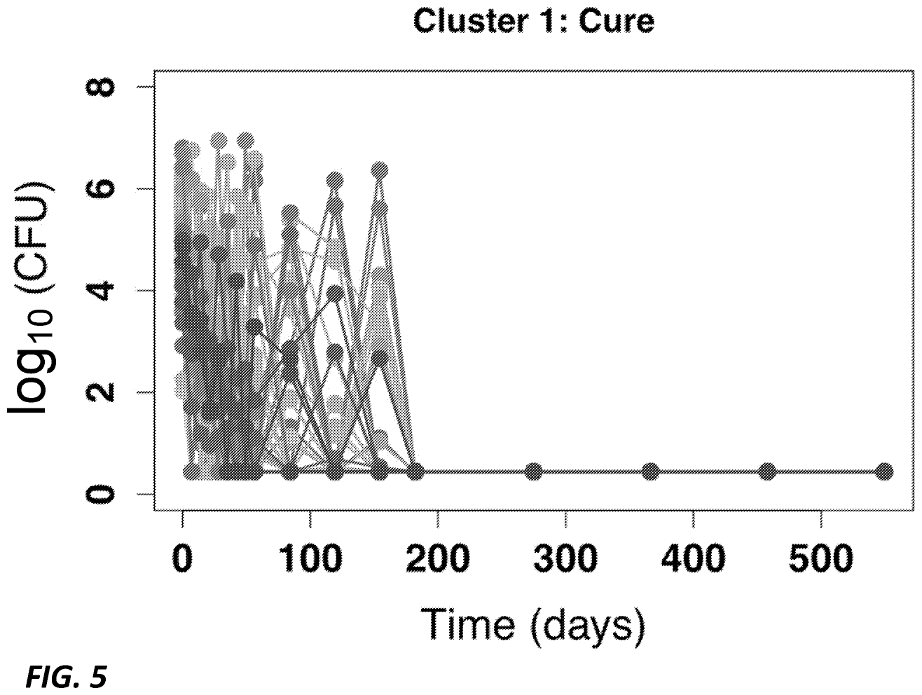

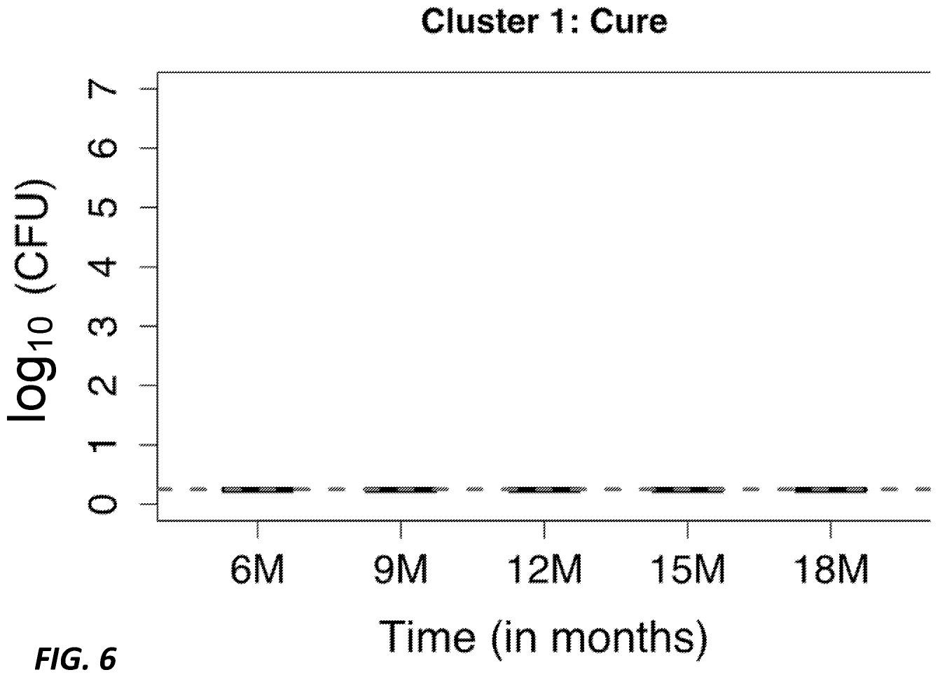

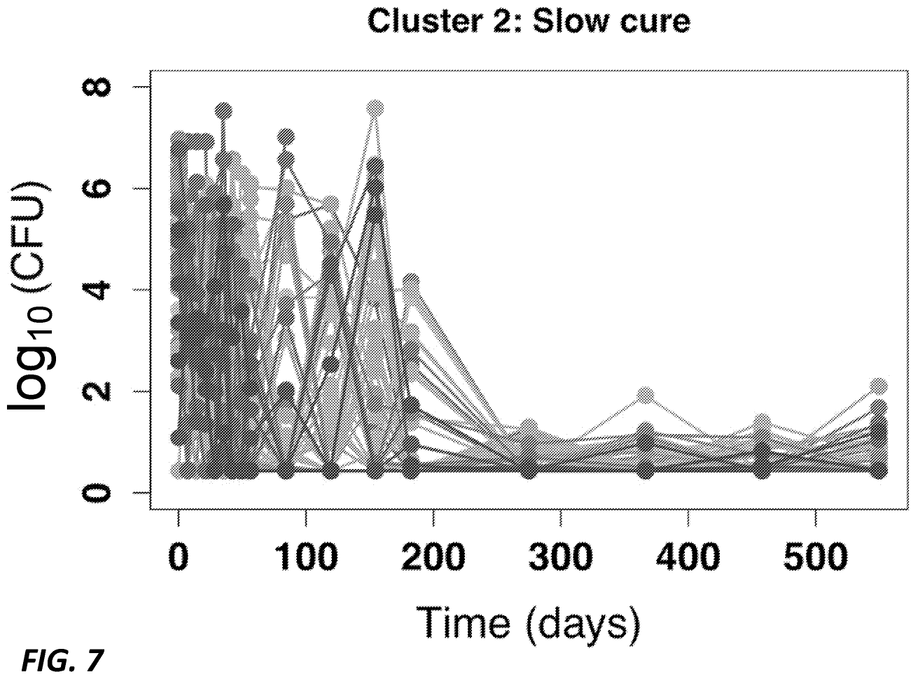

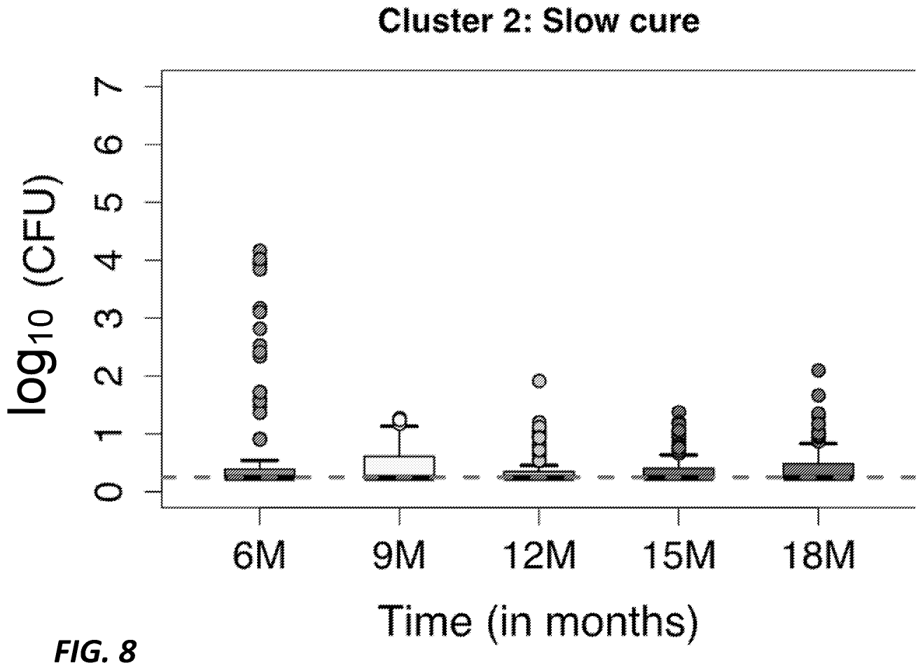

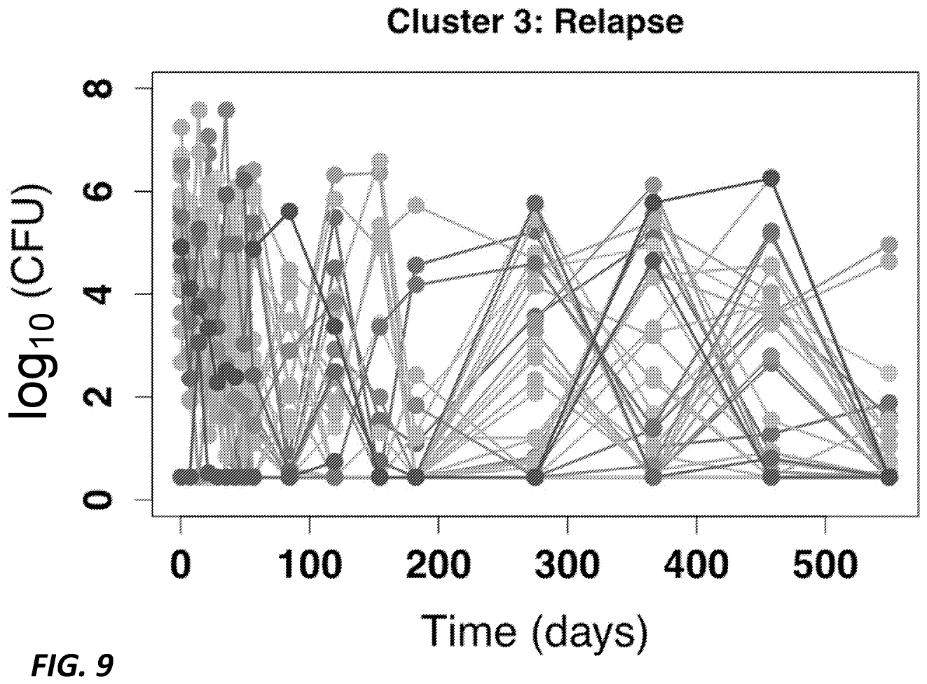

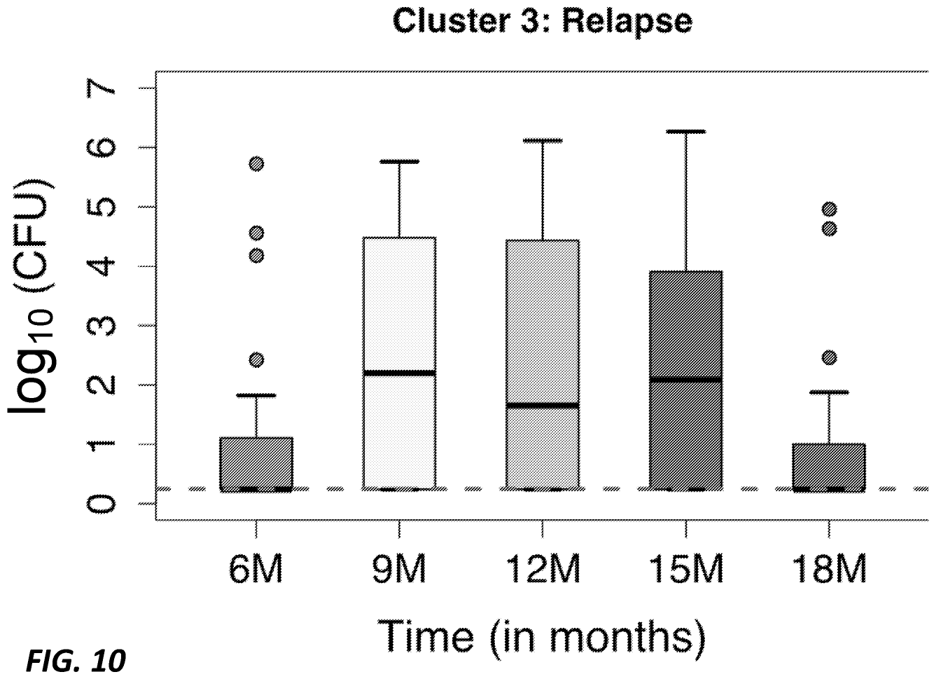

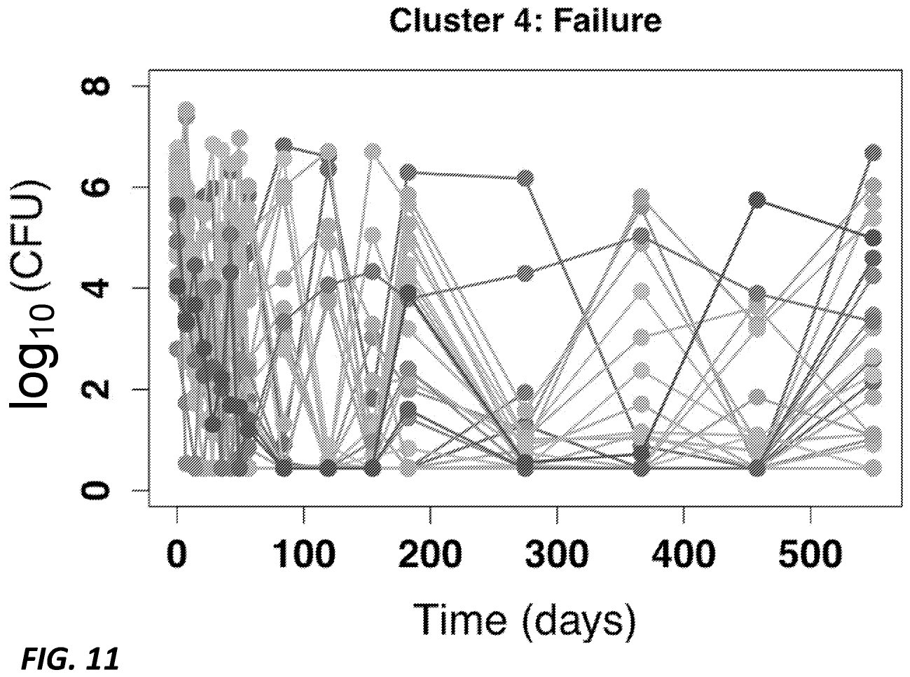

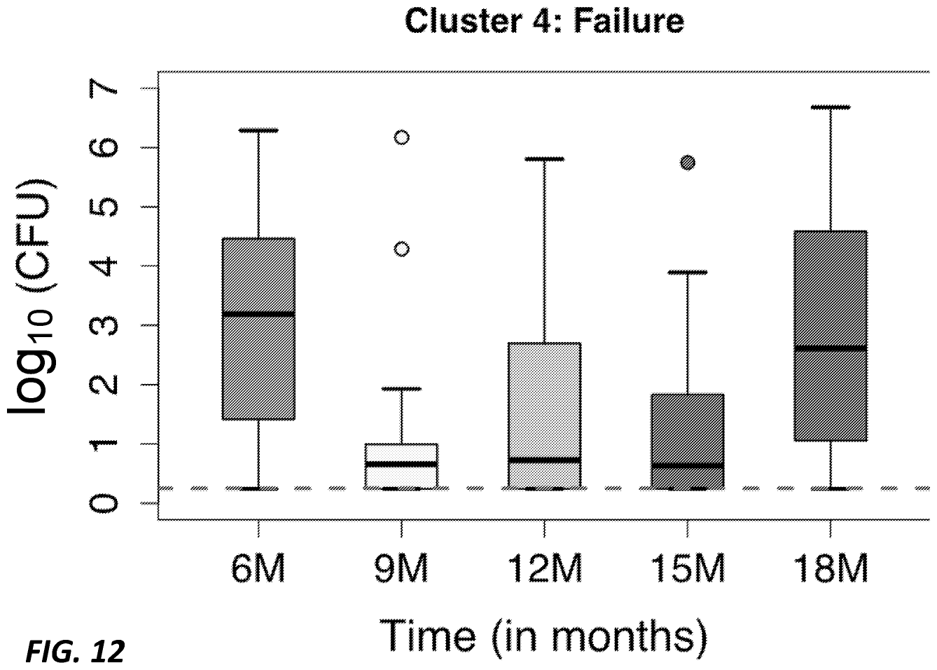

Since the primary predictor was γₛ, such slopes were utilized in a clustering method. Clustering identified four distinct groups based on trajectories and TTE in 238 patients in the derivation dataset, shown in FIGS. 5-12. These were, a [1] cure cluster of 80 (33.61%) patients, [2] slow-cure cluster of 100 (42.02%), [3] disease relapse cluster 34 (14.28%), and [5] treatment failure cluster 24 [10.08%]. These clusters represent 74.84% (238/318) patients with less than 2 or more missing observations during follow up.

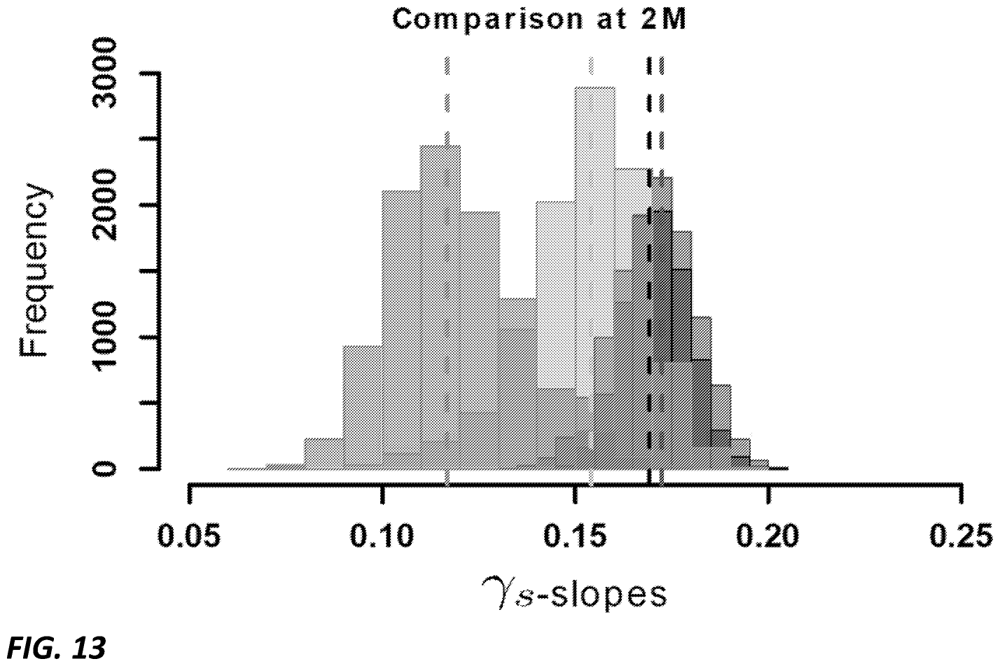

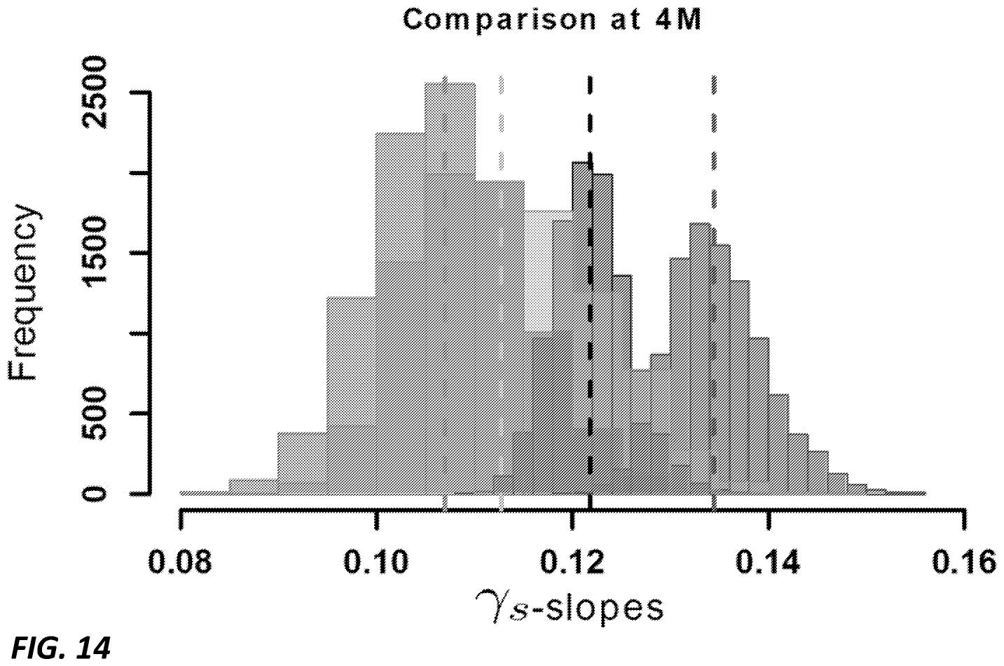

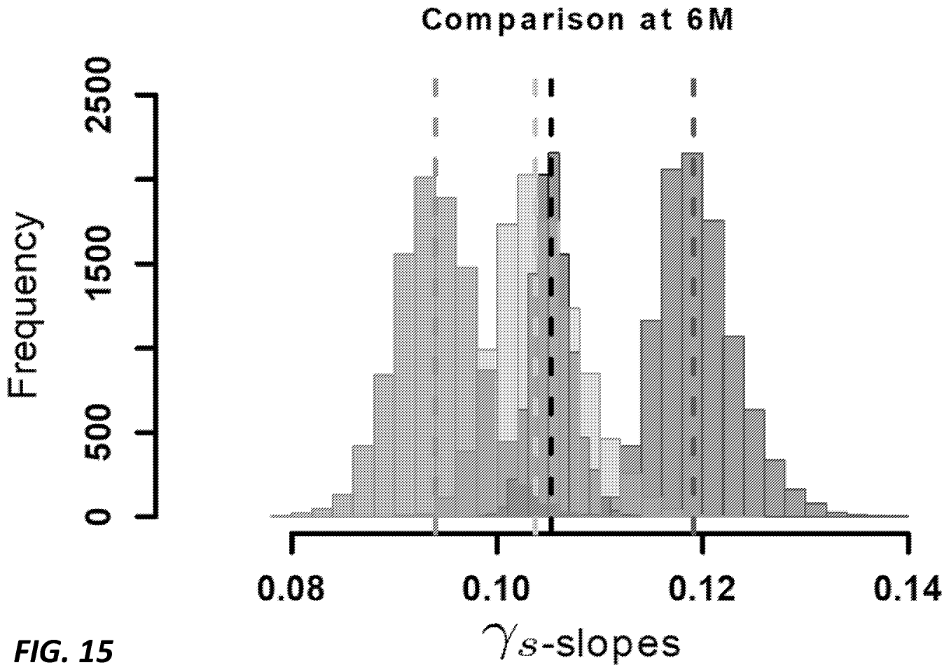

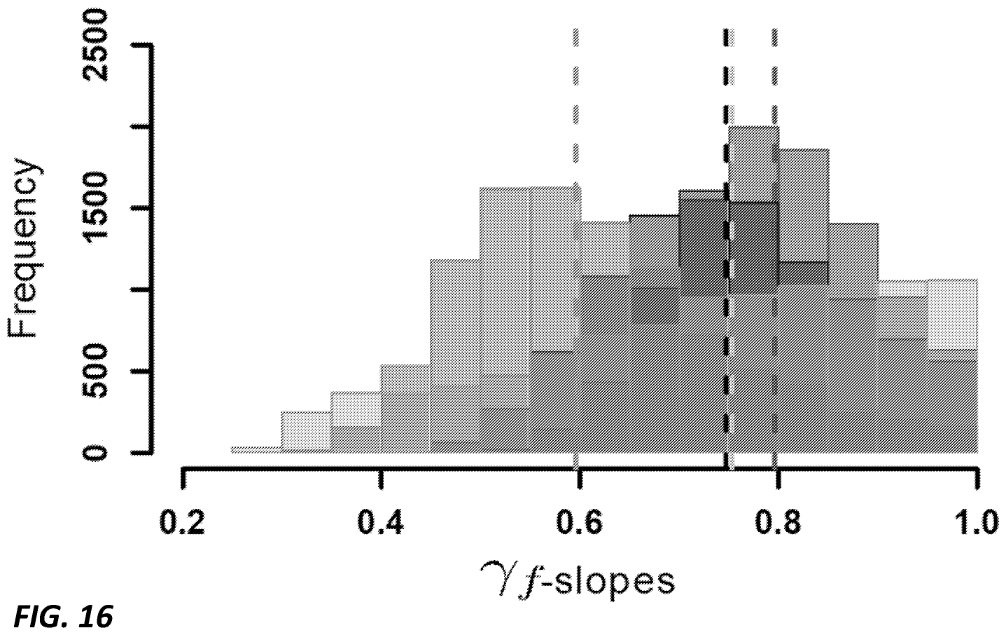

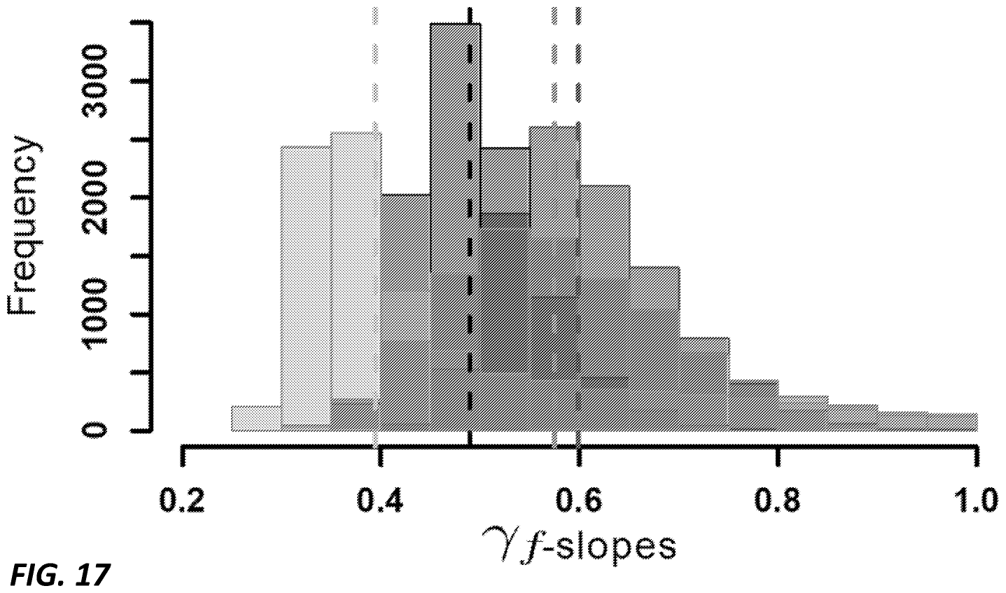

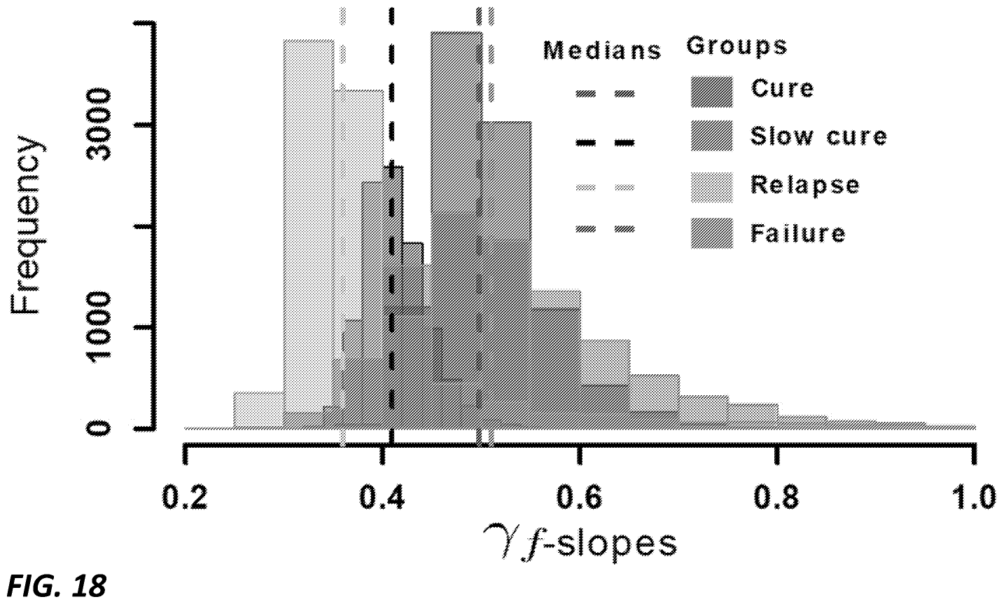

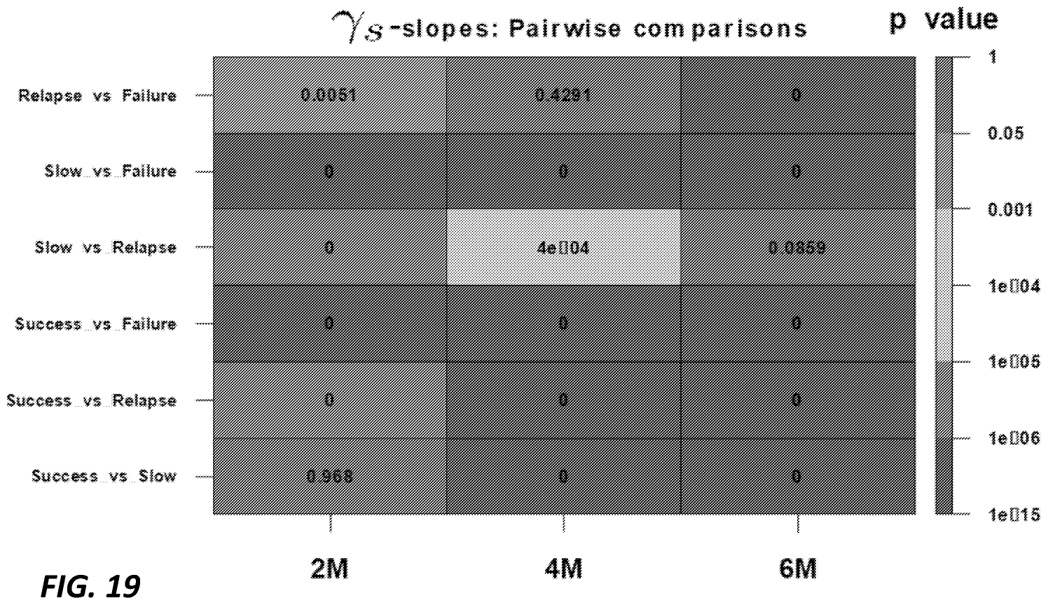

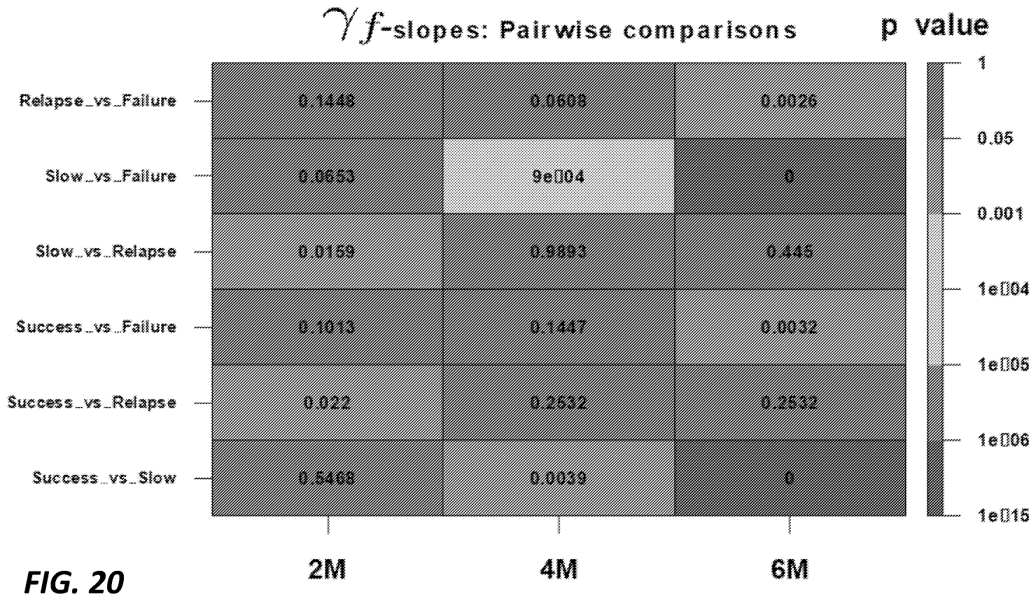

FIGS. 13-20 show the distribution of model derived γₛ and γ_{f} values, including how they change from 2 to 4 months and from 4 to 6 months, and the pairwise comparisons of the relationships between slopes. The Figures show that the γ_{f} values could not discriminate failures from cures. On the other hand, γₛ=0.15 or <0.1 log₁₀ CFU/mL/day (i.e., modeling semi-dormant/non-replicating Mtb) were better at discriminating outcomes. The slopes derived with 2 months-vs-4 months data differed in the misclassification of patients, the former misclassifying relapses as cures and the latter misclassifying more cures as relapses. In addition, there was a significant overlap between the 95% Crls of the slow cure and relapse clusters, and an indeterminate outcome zone between γₛ values of 0.10-0.15 log₁₀ CFU/mL/day.

5. Derivation of Biomarker Thresholds in Indeterminate Zones Using Simulations

Next, both MCMC simulations (with LHS parameters sampling) of TTE as well as CART in R were used to probe and identify γₛ thresholds in the indeterminate outcomes region, with results shown in Table 2. Cure was clearly delineated by γₛ>0.15, failure by γₛ<0.1 plus initial bacterial burden (B(0)>5.6 log₁₀ CFU/mL (TTP=5.49 days), and relapse from cure in the region γₛ<0.13. Patients with initial bacteria burden B(0)>4.5 log₁₀ CFU/mL (TTP=8.11 days), and γₛ-slopes between 0.1 and 0.15 had >55% chance of failing treatment at 6 months. However, for a 4 month duration regimen, patients with B(0)>5.4 log₁₀ CFU/mL (TTP=5.93) and γₛ between 0.09 and 0.14 had a >65% chance of failing treatment. The data indicated that in order to achieve cure within 2 months, then γₛ≥0.15 (−3.90 TTP per day) would be required, while patients with γₛ≤0.1 (−2.60 TTP per day) would fail. Patients on standard therapy with B(0)>5.6 log₁₀ CFU/mL (TTP=5.49) with γₛ<0.13 would relapse.

TABLE 2
Biomarker threshold (cut-off) values

| | | | |
|---|---|---|---|
| | | | Initial |
| Slope evaluate | γₛ cut-off [log10 | γₛ cut-off | bacterial |
| at Month | CFU/mL/day] | [TTP/day] | [TTP/day] |

γₛ Magnitude cut-off values and initial bacteria burden (Cure regions)

| | | | |
|---|---|---|---|
| At 2 Months | >0.15 | −3.90 | — |
| At 4 Months | >0.14 | −3.64 | — |
| At 6 Months | >0.11 | −2.86 | — |

Conditional cure regions

| | | | |
|---|---|---|---|
| At 2 Months | 0.1 < γₛ < 0.15 | −3.9 < γₛ < −2.6 | −<8.11 |
| At 4 Months | 0.09 < γₛ < 0.14 | −2.86 < γₛ < −2.34 | −<5.93 |

Failure region

| | | | |
|---|---|---|---|
| At 2 Months | <0.1 | <−2.60 | — |
| At 4 Months | <0.1 | <−2.60 | — |

Region of which high chance of relapse

| | | | |
|---|---|---|---|
| At 2 and 6 Months | 0.1 < γₛ < 0.15 | −3.9 < γₛ < −2.6 | <−5.49 |

Table 2 provides a summary of cut-off values for the γₛ and initial bacteria burden that can be used in clinical trials to determine patients that will fail treatment and those that will proceed to get cured using biomarkers at 2 and 4 months. At 6 months, slower slopes than the given cut-off discriminates failed treatment outcome from successful outcomes.

6. Sensitivity Analysis of Biomarkers and their Impact on Patient's Time to Cure

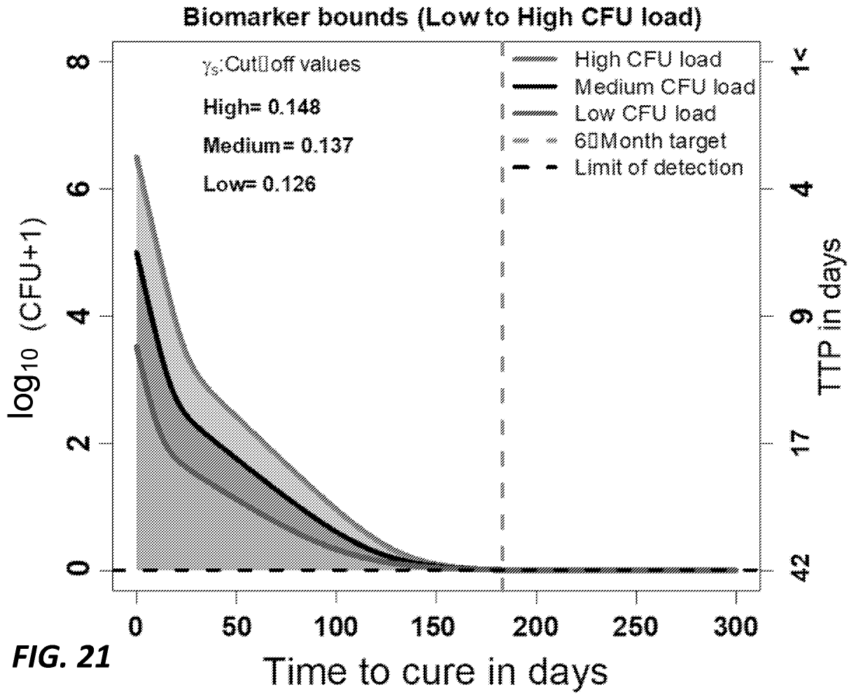

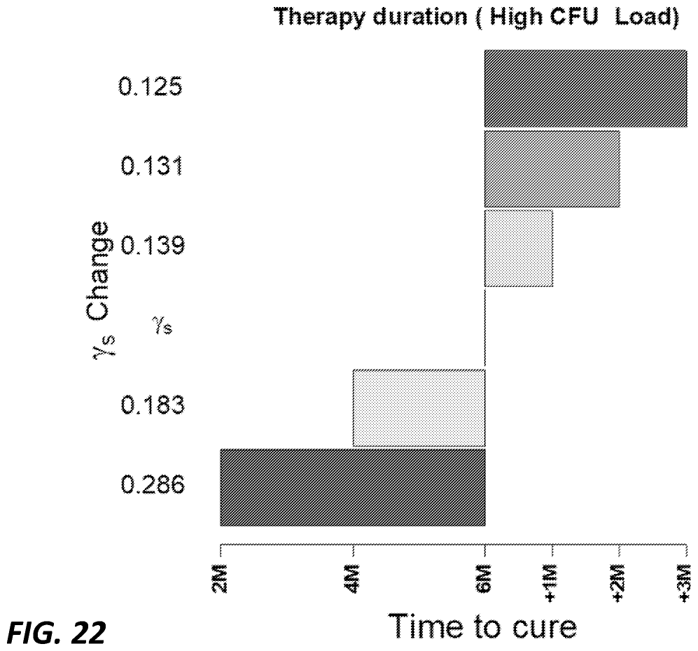

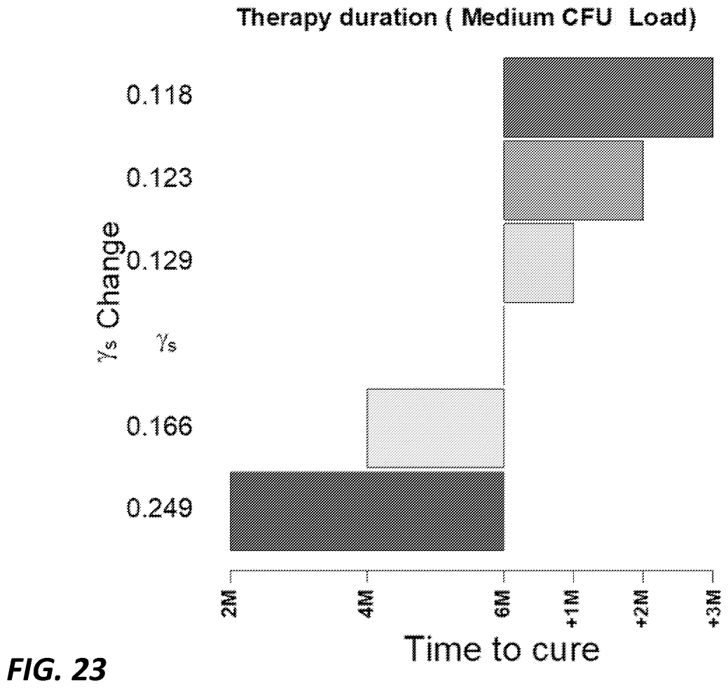

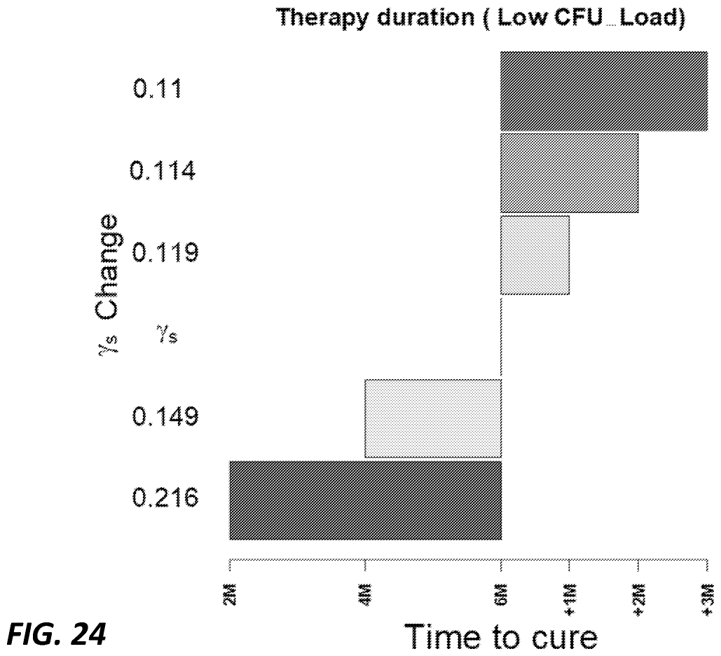

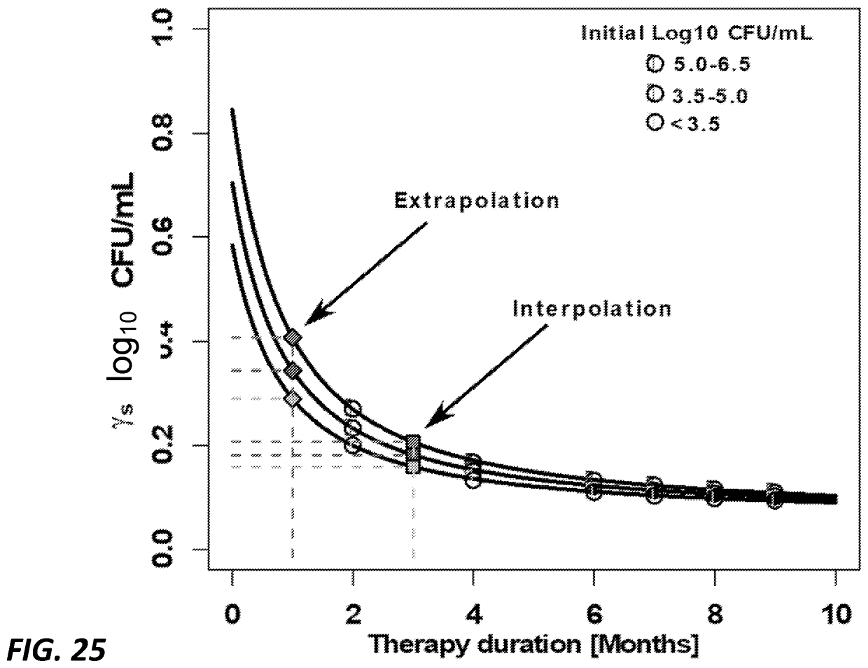

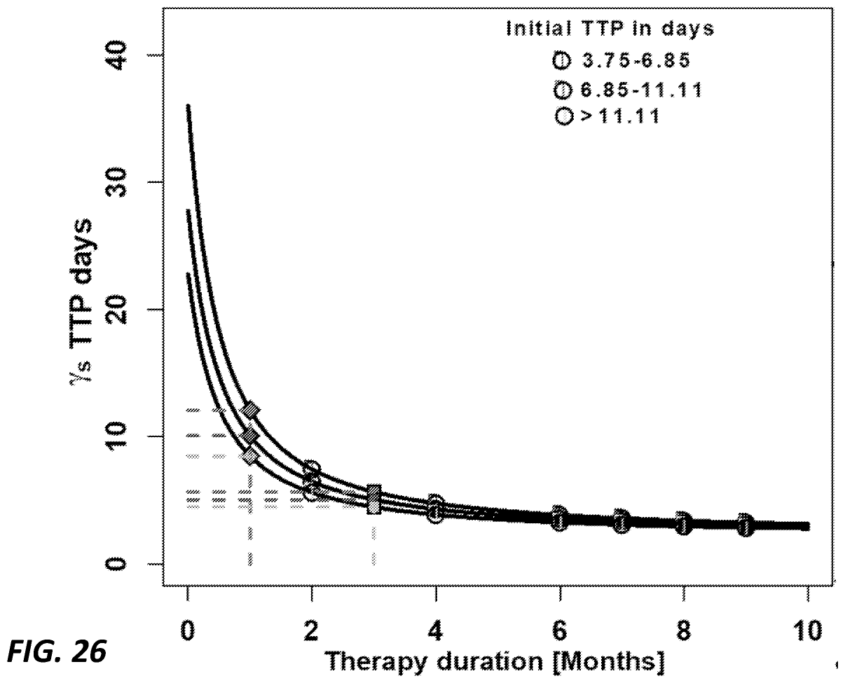

FIGS. 21-26 show sensitivity analysis of the γₛ-slopes as patient treatment duration changed. FIGS. 21-24 show that increasing or reducing the γₛ (i. e., sterilization effect rates) changes time-to-extinction (TTE) and therefore the required minimum duration of therapy. As an example, the 6-months therapy would extend to 8 and 9 months (i.e., slow-cure) in patients with high bacterial burden, when γₛ (=0.148) is reduced to 0.131 and 0.125 (FIG. 22). On the other hand, to reduce treatment duration to 2 and 4 months, γₛ should be increased to 0.286 and to 0.183, respectively (FIG. 22). However, for patients in the medium and low CFU load categories, lower sterilization rates can still achieve cure within 6 months, see FIGS. 23-24. The relationship between γₛ and initial TTP versus minimum duration of therapy are shown in FIGS. 25-26, together with calculated target γₛ for one month duration therapy.

7. Validation of the Characterized Biomarkers and their Predictive Accuracy

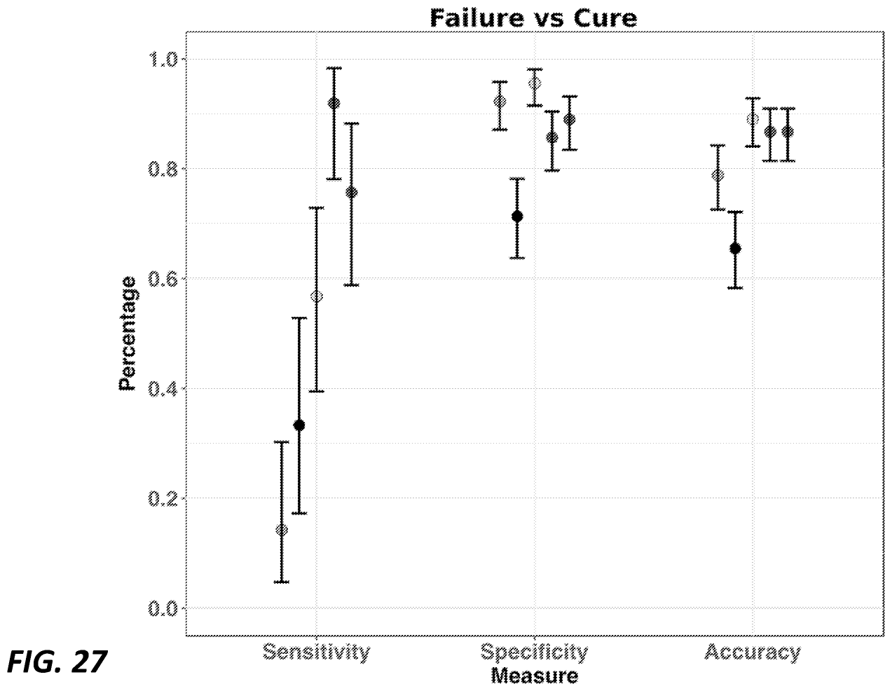

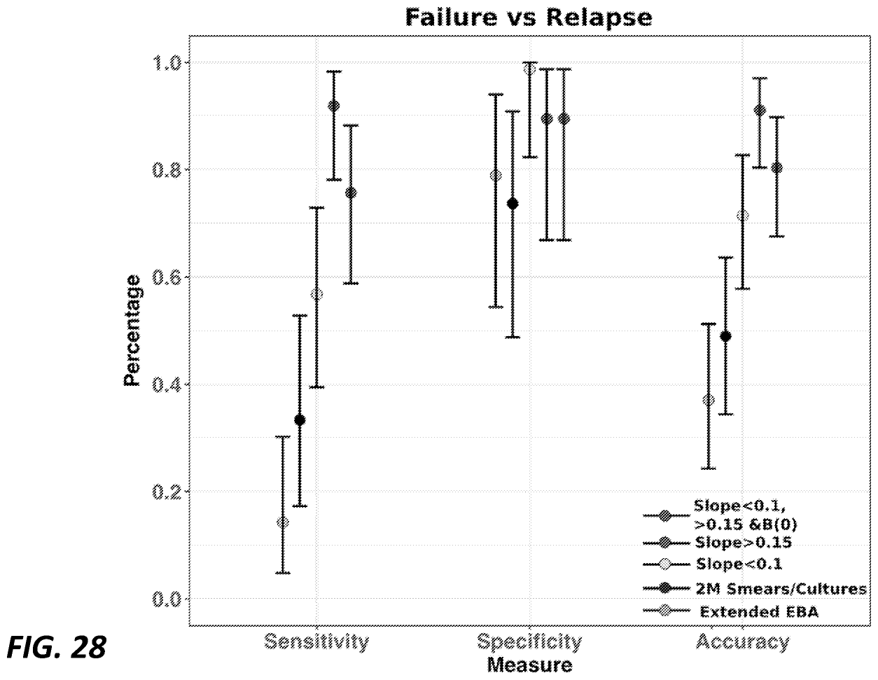

Next, the sensitivity, specificity and accuracy of all the derived 8-week-based biomarkers in the validation data sets were calculated, with results shown in FIGS. 27-28. The γₛ biomarker combined with the initial bacterial burden had a sensitivity of 0.92 and specificity of 0.86 for 6 months therapy duration (validation dataset n=319 patients) on standard therapy, which was better than the sensitivity of 0.14 with specificity of 0.92 for the extended EBA, and a sensitivity of 0.33 with specificity of 0.71 for 2-month smears/cultures. The standard therapy-derived slopes in derivation dataset identified for TTE at 4 months were tested in the validation dataset using the 4-month ReMOX experimental regimens, with results shown in FIGS. 27-28. The sensitivity was 0.81 and the specificity of 0.87 in the isoniazid arm (N=654), while sensitivity was 0.70 the specificity was 0.71 in the ethambutol arm (N=633).

### D. Discussion

The first major implications of our findings are on TB clinical trial design. To begin with the γ_{f}-based indices such as extended EBA demonstrated a poor forecasting accuracy, which suggests that there should be a potential change in the early clinical test strategy used to identify effective regimens. The extended EBA and 2-month smears (which are best described by γ_{f}) had very low sensitivity but good specificity for cure, which means that most regimens with good sterilizing effect would be thrown away (too many false negatives for sterilizing effect) so as to render them counterproductive in regimen selection for sterilizing effect. One potential pathway could be to identify and rank regimens using preclinical models that have good forecasting accuracy. (See Magombedze G et al. (2018); Gumbo T. et al. (J Infect Dis 2015); and Gumbo T et al. (Clin Infect Dis 2015)). The regimens so derived, including optimal doses, which provide good Bayesian priors, can then be tested versus standard therapy in an 8-week clinical trial (Phase II) using weekly TTP as the main output plus measurement of drug concentrations. The γₛ, initial TTP, and trajectories (vector) can then be examined to determine if TTE was going to be achieved by the end of the proposed duration of therapy, and if so the pharmacokinetics used to optimize the dose to that needed to achieve target γₛ and Mtb population extinction in a larger proportion of patients. The TTE-based models allowed identification of target slopes to be achieved by regimens administered for less than the current six months, allowing for decision-making for different therapy durations after the 8-week study. These data, and the variance in slopes, can then be used to calculate sample size and the expected response rates in the experimental regimens in the design of a phase III trial.

The second major implication is that the slopes derived as biomarkers were in the standard therapy arm, and yet showed excellent accuracy for experimental regimens for shorter durations of therapy. Thus, while specificities of different regimens and pharmacokinetic variability drive the magnitude of slopes, the predictive value of the γₛ slope on outcomes is independent of regimen. Indeed, patients were not excluded who could have MDR-TB or monoresistance in our analyses; they would simply have low γₛ on standard therapy. This means that the derived slopes can be used for MDR-TB regimens as well, and indeed for any regimen. As an example, if a novel 4-week regimen were devised, the slope can still be stated that would achieve that, as shown in FIGS. 25-26.

Third, sputum smears and cultures, and 2-month smears/cultures are recommended in routine care in TB programs worldwide, and are used to personalize duration of therapy. The sensitivity and specificity of this test in forecasting long-term outcomes has been subject of considerable debate. (See Horne D J et al. (2010); and Wallis R S et al. (2013)). Here, it has also been found that it has poor forecasting accuracy for therapy failure and relapse in the individual patient. On the other hand, TTP-based slopes and initial bacterial have been found to have higher sensitivity, specificity, and accuracy in identifying patients who would fail therapy or relapse. If these patients with higher rates of therapy failure and potential relapse could be identified during the first 8 weeks of therapy, then interventions such as dose increases or switching therapy regimens, as done in HIV-treatment programs. (See Pasipanodya J G et al. (2013)). On the other hand, these slopes could also be used by the TB program to identify patients who can get away with shorter therapy duration. Since many programs already employ liquid culture systems from which TTP is generated, it means that the biomarker proposed herein would come at no extra cost to the TB program.

Our study has some limitations. First, it could be argued that our findings are specific to the dataset we analyzed. Thus work with other datasets is being pursued. Second, calculation of slopes is relatively complex; however software can easily be written to automate this. Finally, not all patients who do not reach TTE by end of therapy will fail therapy or relapse, thus our approach may lead to over treating of these patients. However, even with these limitations, early TTP-based biomarkers were identified that predict long-term clinical outcomes and relapse for different therapy durations, with high sensitivity and specificity in validation datasets.

### E. Interpretation

We have identified early TTP-based biomarkers to better predict long term clinical outcomes and relapse for different therapy durations.

Although one or more of the disclosed figures may illustrate systems, apparatuses, methods, or a combination thereof, according to the teachings of the disclosure, the disclosure is not limited to these illustrated systems, apparatuses, methods, or a combination thereof. One or more functions or components of any of the disclosed figures as illustrated or described herein may be combined with one or more other portions of another function or component of the disclosed figures. Accordingly, no single implementation described herein should be construed as limiting and implementations of the disclosure may be suitably combined without departing from the teachings of the disclosure.

Although the present disclosure and its advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, and composition of matter, means, methods and steps described in the specification. As one of ordinary skill in the art will readily appreciate from the disclosure, processes, machines, means, methods, or steps, presently existing or later to be developed that perform substantially the same function or achieve substantially the same result as the corresponding embodiments described herein can be utilized according to the present examples. Accordingly, the appended claims are intended to include within their scope such processes, machines, means, methods, or steps.

The claims are not intended to include, and should not be interpreted to include means-plus- or step-plus-function limitations, unless such a limitation is explicitly recited in a given claim using the phrase(s) “means for” or “step for,” respectively.

### REFERENCES

The following references and publications referred to throughout the specification, to the extent that they provide exemplary procedural or other details supplementary to those set forth herein, are specifically incorporated herein by reference.
[1] Dheda K, Gumbo T, Maartens G, et al. The epidemiology, pathogenesis, transmission, diagnosis, and management of multidrug-resistant, extensively drug-resistant, and incurable tuberculosis. Lancet Respir Med 2017.
[2] Gillespie S H, Crook A M, McHugh T D, et al. Four-month moxifloxacin-based regimens for drug-sensitive tuberculosis. N Engl J Med 2014; 371(17): 1577-87.
[3] Rosenthal I M, Zhang M, Williams K N, et al. Daily dosing of rifapentine cures tuberculosis in three months or less in the murine model. PLoSMed 2007; 4(12): e344.
[4] Van D A, Maug A K, Salim M A, et al. Short, highly effective, and inexpensive standardized treatment of multidrug-resistant tuberculosis. AmJ RespirCrit Care Med 2010; 182(5): 684-92.
[5] Tasneen R, Williams K, Amoabeng O, et al. Contribution of the nitroimidazoles PA-824 and TBA-354 to the activity of novel regimens in murine models of tuberculosis. Antimicrobial Agents and Chemotherapy 2014.
[6] Deshpande D, Pasipanodya J G, Srivastava S, et al. Minocycline immunomodulation via sonic hedgehog signaling and apoptosis and direct potency against drug-resistant tuberculosis. Journal of Infectious Diseases 2018; in press.
[7] Deshpande D, Srivastava S, Chapagain M, et al. Ceftazidime-avibactam has potent sterilizing activity against highly drug-resistant tuberculosis. Sci Adv 2017; 3(8): e1701102.
[8] BEST (Biomarkers, EndpointS, and other Tools) Resource. Silver Spring (Md.); 2016.
[9] Burman W J. The hunt for the elusive surrogate marker of sterilizing activity in tuberculosis treatment. Am J Respir Crit Care Med 2003; 167(10): 1299-301.
[10] Home D J, Royce S E, Gooze L, et al. Sputum monitoring during tuberculosis treatment for predicting outcome: systematic review and meta-analysis. Lancet Infect Dis 2010; 10(6): 387-94.
[11] Wallis R S, Wang C, Meyer D, Thomas N. Month 2 culture status and treatment duration as predictors of tuberculosis relapse risk in a meta-regression model. PLoSOne 2013; 8(8): e71116.
[12] Imperial M Z, Nahid P, Phillips P P J, et al. A patient-level pooled analysis of treatment-shortening regimens for drug-susceptible pulmonary tuberculosis. Nat Med 2018; 24(11): 1708-15.
[13] Jindani A, Aber V R, Edwards E A, Mitchison D A. The early bactericidal activity of drugs in patients with pulmonary tuberculosis. AmRevRespirDis 1980; 121(6): 939-49.
[14] Jindani A, Dore C J, Mitchison D A. Bactericidal and sterilizing activities of antituberculosis drugs during the first 14 days. Am J Respir Crit Care Med 2003; 167(10): 1348-54.
[15] Diacon A H, Dawson R, von Groote-Bidlingmaier F, et al. 14-day bactericidal activity of PA-824, bedaquiline, pyrazinamide, and moxifloxacin combinations: a randomised trial. Lancet 2012; 380(9846): 986-93.
[16] Conde M B, Efron A, Loredo C, et al. Moxifloxacin versus ethambutol in the initial treatment of tuberculosis: a double-blind, randomised, controlled phase II trial. Lancet 2009; 373(9670): 1183-9.
[17] Mellors J W, Rinaldo C R, Jr., Gupta P, White R M, Todd J A, Kingsley L A. Prognosis in HIV-1 infection predicted by the quantity of virus in plasma. Science 1996; 272(5265): 1167-70.
[18] Epstein M D, Schluger N W, Davidow A L, Bonk S, Rom W N, Hanna B. Time to detection of Mycobacterium tuberculosis in sputum culture correlates with outcome in patients receiving treatment for pulmonary tuberculosis. Chest 1998; 113(2): 379-86.
[19] Hesseling A C, Walzl G, Enarson D A, et al. Baseline sputum time to detection predicts month two culture conversion and relapse in non-HIV-infected patients. IntJ TubercLung Dis 2010; 14(5): 560-70.
[20] Pasipanodya J G, Mubanga M, Ntsekhe M, et al. Tuberculous Pericarditis is Multibacillary and Bacterial Burden Drives High Mortality. Ebiomedicine 2015; 2(11): 1634-9.
[21] Dheda K, Lenders L, Magombedze G, et al. Drug-Penetration Gradients Associated with Acquired Drug Resistance in Patients with Tuberculosis. Am J Respir Crit Care Med 2018; 198(9): 1208-19.
[22] Magombedze G, Pasipanodya J G, Srivastava S, et al. Transformation Morphisms and Time-to-Extinction Analysis That Map Therapy Duration From Preclinical Models to Patients With Tuberculosis: Translating From Apples to Oranges. Clin Infect Dis 2018; 67(suppl_3): S349-S58.
[23] Gillespie S H, Gosling R D, Charalambous B M. A reiterative method for calculating the early bactericidal activity of antituberculosis drugs. Am J Respir Crit Care Med 2002; 166(1): 31-5.
[24] Swaminathan S, Pasipanodya J G, Ramachandran G, et al. Drug Concentration Thresholds Predictive of Therapy Failure and Death in Children With Tuberculosis: Bread Crumb Trails in Random Forests. Clin Infect Dis 2016; 63(suppl 3): S63-S74.
[25] Pasipanodya J G, Mcllleron H, Burger A, Wash P A, Smith P, Gumbo T. Serum drug concentrations predictive of pulmonary tuberculosis outcomes. J Infect Dis 2013; 208(9): 1464-73.
[26] Genolini C, Falissard B. KmL: a package to cluster longitudinal data. Comput Methods Programs Biomed 2011; 104(3): e112-21.
[27] Magombedze G, Shiri T, Eda S, Stabel J R. Inferring biomarkers for Mycobacterium avium subsp. paratuberculosis infection and disease progression in cattle using experimental data. Sci Rep 2017; 7: 44765.
[28] Soetaert K, Pedzoldt, T. Inverse modelling, sensitivity and Monte Carlo analysis in R using FME. Journal of Statistical Software 2010; 33(3): 1-28.
[29] Gumbo T, Lenaerts A J, Hanna D, Romero K, Nuermberger E. Nonclinical models for antituberculosis drug development: a landscape analysis. J Infect Dis 2015; 211 Suppl 3: S83-95.
[30] Gumbo T, Pasipanodya J G, Romero K, Hanna D, Nuermberger E. Forecasting Accuracy of the Hollow Fiber Model of Tuberculosis for Clinical Therapeutic Outcomes. Clin Infect Dis 2015; 61 Suppl 1: S25-31.

1. A method for treating a patient with tuberculosis, the method comprising:
receiving patient data corresponding to a TB patient, the patient data associated with sputum time-to-positivity (TTP) data associated with a time period;
identifying, based on the patient data, a kill rate of semidormant/persistent (γₛ) Mycobacterium tuberculosis;
determining a treatment response prediction result based on the kill rate; and
generating an output based on the treatment response prediction result.

2. The method of claim 1, where the time period is less than or equal to six months.

3. The method of claim 1, where the time period is less than or equal to four months.

4. The method of claim 1, where the time period is less than or equal to eight weeks.

5. The method of any of claims 1-4, where the treatment response prediction result is determined from the group consisting of: treatment failure, cure—but will relapse, cure without relapse, and slow-cure.

6. The method of any of claims 1-5, where the patient data includes a TTP value, a colony forming unit (CFU) count/mL, the kill rate of the semidormant/persistent (γₛ) Mycobacterium tuberculosis, an initial bacterial burden, or a combination thereof.

7. The method of any of claims 1-6, further comprising:
calculating the kill rate of the semidormant/persistent (γₛ) Mycobacterium tuberculosis based on the patient data.

8. The method of claim 7, where calculating the kill rate of the semidormant/persistent (γₛ) Mycobacterium tuberculosis comprises:
converting the TTP data to a colony forming unit (CFU) count/mL; and
determining the kill rate as log₁₀ CFU/ml/day for semidormant/persistent (γₛ) Mycobacterium tuberculosis.

9. The method of any of claims 1-8, further comprising, when the patient data corresponds to a time period of 2 months of treatment:
determining, based on the patient data, whether the kill rate of the semidormant/persistent (γₛ) Mycobacterium tuberculosis is greater than or equal to 0.15; and
where the treatment response prediction result comprises cure without relapse in response to a determination that the kill rate of the semidormant/persistent (γₛ) Mycobacterium tuberculosis is greater than or equal to 0.15.

10. The method of any of claims 1-8, further comprising, when the patient data corresponds to a time period of 4 months of treatment:
determining, based on the patient data, whether the kill rate of the semidormant/persistent (γₛ) Mycobacterium tuberculosis is greater than or equal to 0.14; and
where the treatment response prediction result comprises cure without relapse in response to a determination that the kill rate of the semidormant/persistent (γₛ) Mycobacterium tuberculosis is greater than or equal to 0.14.

11. The method of any of claims 1-8, further comprising, when the patient data corresponds to a time period of 6 months of treatment:
determining, based on the patient data, whether the kill rate of the semidormant/persistent (γₛ) Mycobacterium tuberculosis is greater than or equal to 0.11; and
where the treatment response prediction result comprises cure without relapse in response to a determination that the kill rate of the semidormant/persistent (γₛ) Mycobacterium tuberculosis is greater than or equal to 0.11.

12. The method of any of claims 1-8, further comprising, when the patient data corresponds to a time period of 2 or 4 months of treatment:
determining, based on the patient data, whether the kill rate of the semidormant/persistent (γₛ) Mycobacterium tuberculosis is less than or equal to 0.1.

13. The method of claim 12, where the treatment response prediction result comprises treatment failure in response to a determination that the kill rate of the semidormant/persistent (γₛ) Mycobacterium tuberculosis is less than or equal to 0.1.

14. The method of any of claims 1-8, further comprising, when the patient data corresponds to a time period of 2 months of treatment:
determining, based on the patient data, whether the kill rate of the semidormant/persistent (γₛ) Mycobacterium tuberculosis is greater than or equal to 0.1 and less than or equal to 0.15; and
determining, based on the patient data, whether an initial bacterial burden (B(0)) is greater than or equal to 4.5 log₁₀ CFU/mL (TTP=8.11 days); and
where the treatment response prediction result comprises cure—but will relapse in response to a determination that the kill rate of the semidormant/persistent (γₛ) Mycobacterium tuberculosis is between 0.1 and 0.15 and a determination that the initial bacterial burden (B(0)) is greater than or equal to 4.5 log₁₀ CFU/mL (TTP=8.11 days).

15. The method of any of claims 1-8, further comprising, when the patient data corresponds to a time period of 4 months of treatment:
determining, based on the patient data, whether the kill rate of the semidormant/persistent (γₛ) Mycobacterium tuberculosis is greater than or equal to 0.09 and less than or equal to 0.14; and
determining, based on the patient data, whether an initial bacterial burden (B(0)) is greater than or equal to 5.4 log₁₀ CFU/mL (TTP=5.93 days); and
where the treatment response prediction result comprises cure—but will relapse in response to a determination that the kill rate of the semidormant/persistent (γₛ) Mycobacterium tuberculosis is between 0.09 and 0.14 and a determination that the initial bacterial burden (B(0)) is greater than or equal to 5.4 log₁₀ CFU/mL (TTP=5.93 days).

16. The method of any of claims 1-8, further comprising, when the patient data corresponds to a time period of 2 or 6 months of treatment:
determining, based on the patient data, whether the kill rate of the semidormant/persistent (γₛ) Mycobacterium tuberculosis is greater than or equal to 0.1 and less than or equal to 0.15; and
determining, based on the patient data, whether an initial bacterial burden (B(0)) is greater than or equal to 5.6 log₁₀ CFU/mL (TTP=5.49 days); and
where the treatment response prediction result comprises cure—but will relapse in response to a determination that the kill rate of the semidormant/persistent (γₛ) Mycobacterium tuberculosis is between 0.1 and 0.15 and a determination that the initial bacterial burden (B(0)) is greater than or equal to 5.6 log₁₀ CFU/mL (TTP=5.49 days).

17. The method of any of claims 1-16, where the output indicates the treatment response prediction result.

18. The method of any of claims 1-17, further comprising determining a treatment recommendation based on the treatment predication result.

19. The method of claim 18, where the output indicates the treatment recommendation.

20. The method of any of claims 1-19, where, when the treatment response prediction result is cure without relapse, the treatment recommendation indicates a shorter treatment duration.

21. The method of any of claims 1-19, where, when the treatment response prediction result is treatment failure, the treatment recommendation indicates dose increase of one or more anti-TB drugs.

22. The method of any of claims 1-19, where, when the treatment response prediction result is treatment failure, the treatment recommendation indicates to switch a treatment regimen.

23. The method of any of claims 1-19, where, when the treatment response prediction result is cure—but will relapse, the treatment recommendation indicates a longer treatment duration.

24. The method of any of claims 1-19, where, when the treatment response prediction result is cure—but will relapse, the treatment recommendation indicates dose increase of one or more anti-TB drugs.

25. The method of any of claims 1-19, where, when the treatment response prediction result is cure—but will relapse, the treatment recommendation indicates to switch a treatment regimen.

26. A system for treating a patient with tuberculosis, the system comprising:
a receiver configured to receive patient data corresponding to a TB patient, the patient data associated with sputum time-to-positivity (TTP) data associated with a time period; and
a processor coupled to the receiver and configured to:
identify, based on the patient data, a kill rate of semidormant/persistent (γₛ) Mycobacterium tuberculosis;
determine a treatment response prediction result based on the kill rate; and
generate an output based on the treatment response prediction result.

27. The system of claim 26, further comprising a memory coupled to the processor, the memory configured to store one or more instructions executable by the processor to perform one or more operations.

28. The system of any of claims 26-27, where the memory is configured to store at least one threshold value.

29. The system of any of claims 26-28, where the processor is further configured to:
identify a treatment duration associated with the patient data;
retrieve one or more threshold values from the memory base on the treatment duration; and
compare the kill rate to the one or more threshold values to determine the treatment response prediction result.

30. The system of any of claims 26-29, where the processor is further configured to:
determine an initial bacterial burden (B(0)) based on the patient data;
identify a treatment duration associated with the patient data; and
retrieve one or more threshold values from the memory base on the treatment duration; and
compare the initial bacterial burden (B(0)) to the one or more threshold values to determine the treatment response prediction result.

31. The system of any of claims 26-29, where the processor is further configured to:
identify a TTP value based on the patient data; and
calculate an initial bacterial burden (B(0)) based on the TTP value; and
where the treatment response prediction result is further determined based on the initial bacterial burden (B(0)).

32. The system of any of claims 26-31, further comprising a display device coupled to the processor and configured to provide a presentation based on the output.

33. The system of any of claims 26-32, further comprising a transmitter configured to send the output.

34. A non-transitory computer readable medium comprising instructions that, when executed by a processor, cause the processor to:
identify, based on patient data corresponding to a TB patient, a kill rate of semidormant/persistent (γₛ) Mycobacterium tuberculosis, the patient data associated with sputum time-to-positivity (TTP) data associated with a time period;
determine a treatment response prediction result based on the kill rate; and
generate an output based on the treatment response prediction result.

35. A non-transitory computer readable medium comprising instructions that, when executed by a processor, cause the processor to:
identify, based on patient data corresponding to a TB patient, an initial bacterial burden (B(0)) associated with Mycobacterium tuberculosis, the initial bacterial burden (B(0)) associated with a sputum time-to-positivity (TTP) value;
determine a treatment response prediction result based on the initial bacterial burden (B(0)); and
generate an output based on the treatment response prediction result.

36. A method for treating a patient with tuberculosis, the method comprising:
receiving patient data corresponding to a TB patient;
identifying, based on the patient data, a concentration of an anti-TB drug in a patient;
performing a comparison between the concentration and one or more thresholds; and
generating an output based on the comparison.

37. The method of claim 36, where the anti-TB drug includes isoniazid, rifampin, pyrazinamide, ethambutol, levofloxacin, gatifloxacin, amikacin, ethionamide, or cycloserine.

38. The method of any of claims 36-37, where the output indicates to increase a dose of the anti-TB drug, an amount to adjust the dose of the anti-TB drug, or both.

39. The method of any of claims 36-37, where the output indicates a toxicity condition associated with the concentration of the anti-TB drug in the patient.

40. The method of claim 39, where the output indicates to reduce a dose of the anti-TB drug based on the toxicity condition.

41. The method of any of claims 36-40, further comprising identifying a current dose that patient is receiving.

42. The method of any of claims 36-41, further comprising identifying at least one clinical characteristics associated with the patient.

43. The method of claim 42, where the at least one clinical characteristic includes weight, biological sex, height, and tuberculosis site of the patient.

44. The method of any of claims 36-43, where the output indicates a time to perform a next measurement of drug concentration.
